(19) United States
(12) Patent Application Publication
(10) Pub. No.: US 2004/0224954 A1
(43) Pub. Date: Nov. 11, 2004

(54) Substituted 1H-quinoxalin-2-one compounds and substituted 4-aryl- and 4-heteroarylcyclohexane compounds

(76) Inventors: Michael Sattlegger, Bonn (DE); Helmut Buschmann, Esplugues de Llobregat (ES); Michael Przewosny, Aachen (DE); Werner Englberger, Stolberg (DE); Babette-Yvonne Koegel, Langerwehe-Hamich (DE); Hans Schick, Berlin (DE)

Correspondence Address: Geza C. Ziegler, Jr., PERMAN & GREEN, LLP, 425 Post Road, Fairfield, CT, 06824, US

(21) Appl. No.: 10/832,205

(22) Filed: Apr. 26, 2004

Related U.S. Application Data
(63) Continuation of application No. PCT/EP02/11832, filed on Oct. 23, 2002.

(30) Foreign Application Priority Data
Oct. 29, 2001 (DE) 101 53 345.4

(51) Int. Cl. A61K 31/498; C07D 241/36

(52) U.S. Cl. 514/249; 544/354

(57) ABSTRACT

The invention relates to substituted (1H)quinoxalin-2-one compounds, methods for production thereof, medicaments containing said compounds and the use of said compounds for the production of medicaments. The invention further relates to substituted 4-aryl- and 4-heteroarylcyclohexane compounds and methods for production thereof.

The present invention relates to substituted 1H-quinoxalin-2-one compounds, to a process for the production thereof, to pharmaceutical preparations containing these compounds and to the use of these compounds for the production of pharmaceutical preparations and to substituted 4-aryl- and 4-heteroarylcyclohexane compounds and to a process for the production thereof.

The treatment of pain is of great medical significance. There is a worldwide need for effective pain treatments. The urgency of the requirement for effective therapeutic methods for providing tailored and targeted treatment of chronic and non-chronic pain, this being taken to mean pain treatment which is effective and satisfactory from the patient's standpoint, is evident from the large number of scientific papers relating to applied analgesia and to basic nociception research which have appeared in recent times.

Conventional opioids, such as for example morphine, are effective in the treatment of severe to very severe pain. However, they produce unwanted accompanying symptoms which include respiratory depression, vomiting, sedation, constipation and development of tolerance. Moreover, they are less effective in treating neuropathic or incidental pain, which is in particular frequently experienced by tumour patients.

The object of the present invention was accordingly to provide new compounds which are suitable as pharmaceutical active ingredients in pharmaceutical preparations, preferably as pharmaceutical active ingredients for combatting pain, preferably chronic or neuropathic pain and may be used for the treatment or prevention of neurodegenerative diseases, preferably Alzheimer's disease, Huntington's chorea or Parkinson's disease, stroke, cerebral infarct, cerebral ischaemia, cerebral oedema, insufficiency states of the central nervous system, preferably hypoxia or anoxia, epilepsy, schizophrenia, psychoses brought about by elevated amino acid levels, AIDS dementia, encephalomyelitis, Tourette's syndrome, perinatal asphyxia, tinnitus, migraine, inflammatory and/or allergic reactions, depression, mental health conditions, urinary incontinence, pruritus or diarrhoea or for anxiolysis or anaesthesia.

According to the invention, this object is achieved by the provision of substituted 1H-quinoxalin-2-one compounds of the general formula I below and the tautomers thereof, optionally in the form of the diastereomers, pure enantiomers, racemates, non-racemic mixtures of enantiomers or diastereomers and in each case optionally in the form of corresponding bases, salts and solvates, wherein these compounds exhibit in particular an excellent analgesic action.

The present invention accordingly provides substituted 1H-quinoxalin-2-one compounds of the general formula I and the tautomers thereof,

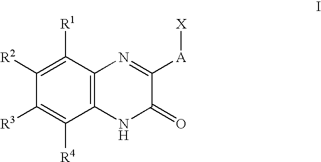

in which

R¹, R², R³ and R⁴, identical or different, denote a linear or branched, saturated or unsaturated aliphatic C₁₋₁₀ residue or a saturated or unsaturated cycloaliphatic C₃₋₇ residue, wherein each of the above-stated residues may optionally be joined together via an ether bridge, or hydrogen, a halogen or a hydroxy group,

A denotes a bridge with one of the following formulae: —(CH₂)ₙ₊₂—, —(CH₂)ₙ—CH═CH—, —(CH₂)ₙCOO—, —(CH₂)ₙCONH—, —(CH₂)ₙ₊₁O(CH₂)ₚCO—, —(CH₂)ₙ₊₁, O—, —(CH₂)ₙ₊₁NR^{1′}—, —NH—(CH₂)ᵣ—, in which n denotes 0, 1, 2 or 3, p denotes 0 or 1 and r denotes 0, 1 or 2, R^{1′} has the meaning stated hereinafter and the bond to the residue X is always stated last and wherein bonding of the residues X¹⁷ and X¹⁶ is possible only via the three bridges stated first and bonding of the residue X⁷ via an amide bridge is excepted,

and X denotes one of the following residues of the general formulae X¹ to X₁₈, in which the unoccupied bond line symbolises the bond to the bridge A and

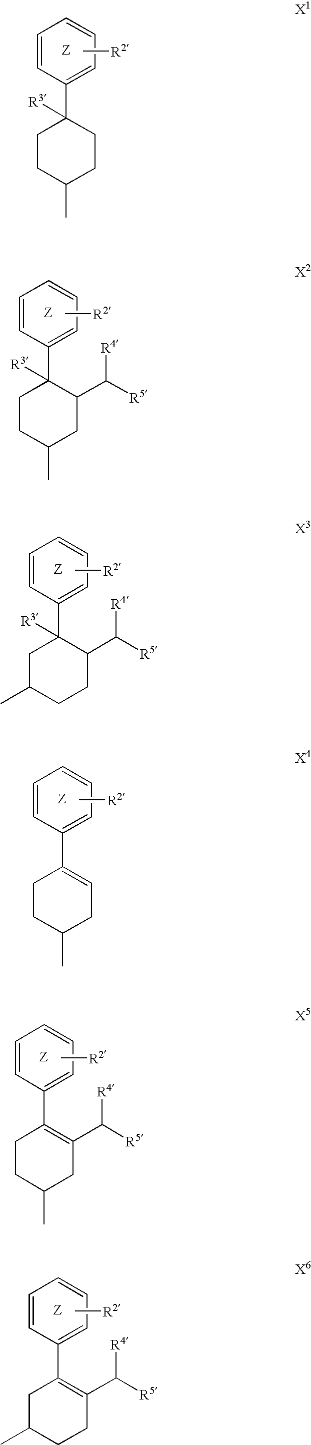

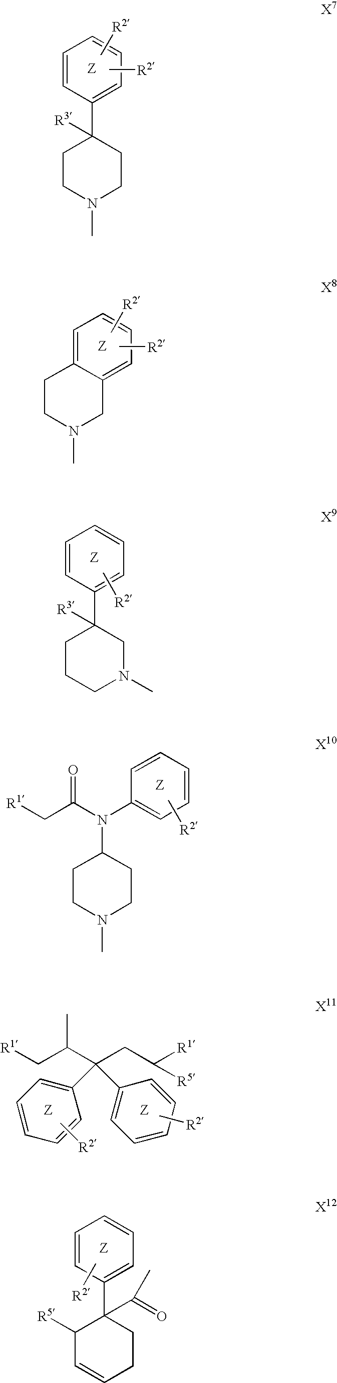

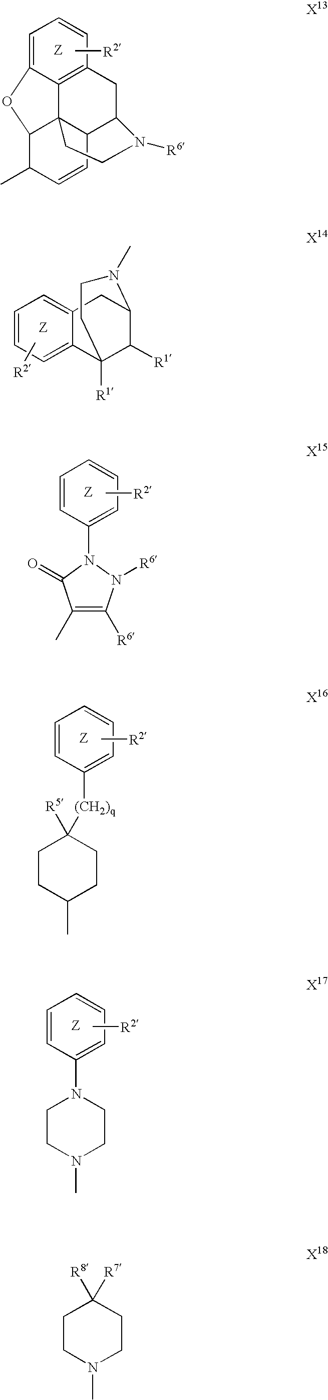

in which

R^{1′} denotes hydrogen, a linear or branched, saturated or unsaturated aliphatic C₁₋₁₀ residue, a saturated or unsaturated cycloaliphatic C₃₋₇ residue, an aryl or heteroaryl residue,

R^{2′} denotes a linear or branched, saturated or unsaturated aliphatic C₁₋₁₀ residue, a saturated or unsaturated cycloaliphatic C₃₋₇ residue or an aryl- or heteroaryl residue, wherein all the above-stated residues may optionally be joined via an ether, thioether or SO₂ bridge, or hydrogen, a halogen, a hydroxy, thiol, cyano or nitro group or a group of the formula —CH₂F, —CHF₂, —CF₃ or —NR^{1′}₂, wherein the two residues R^{1′} are identical or different and have the above-stated meaning,

R^{3′} denotes a linear or branched, saturated or unsaturated aliphatic C₁₋₁₀ residue, a saturated or unsaturated cycloaliphatic C₃₋₇ residue, an aryl or heteroaryl residue, wherein all the above-stated residues may optionally be joined via an ether or an ester bridge, hydrogen, a halogen, a hydroxy group,

R^{4′} denotes hydrogen, an aryl or heteroaryl residue, wherein the aryl or heteroaryl residue may comprise at least one substituent R^{2′} with the above meaning, with the exception of hydrogen,

R^{5′} denotes a residue of the formula —NR^{6′}₂, wherein the two residues R^{6′} may be identical or different and have the meaning stated hereinafter or may form a 3-7-membered ring together with the nitrogen atom connecting them as a ring member, which ring may optionally contain at least one oxygen and/or at least one further nitrogen as a ring atom, wherein the nitrogen may comprise a substituent R^{10′} with the meaning stated hereinafter,

R^{6′} denotes a linear or branched, saturated or unsaturated aliphatic C₁₋₆ residue, a saturated or unsaturated cycloaliphatic C₃₋₇ residue, an aryl or heteroaryl residue,

R^{7′} denotes a cyano, amide or carboxylic acid residue,

R^{8′} denotes a residue of the formula —NR^{9′}₂, wherein the two residues R9′ may be identical or different and have the meaning stated hereinafter or may form a 3-7-membered ring together with the nitrogen atom connecting them as a ring member, which ring may optionally contain at least one oxygen and/or at least one further nitrogen as a ring atom,

R^{9′} denotes hydrogen, a linear or branched aliphatic C₁₋₁₀ residue,

R^{10′} denotes hydrogen, a linear or branched, saturated or unsaturated aliphatic C₁₋₁₀ residue, an aryl or heteroaryl residue and

Z denotes at least one optionally present oxygen, sulfur or nitrogen as a ring atom,

and q denotes 0, 1, 2 or 3,

optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Preferred substituted 1H-quinoxalin-2-one compounds of the general formula I and the tautomers thereof are those in which R² and R³, identical or different, denote a linear or branched, saturated or unsaturated aliphatic C₁₋₃ residue or a halogen and R¹ and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Preferred substituted 1H-quinoxalin-2-one compounds of the general formula I and the tautomers thereof are also those in which R³ denotes a linear or branched, saturated or unsaturated aliphatic C₁₋₃ residue or a halogen and R¹, R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Preferred substituted 1H-quinoxalin-2-one compounds of the general formula I and the tautomers thereof are also those in which R¹ and R³, identical or different, denote a linear or branched, saturated or unsaturated aliphatic C₁₋₃ residue or a halogen and R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Particularly preferred substituted 1H-quinoxalin-2-one compounds of the general formula I and the tautomers thereof are those in which R² and R³ in each case denote a methyl group or a chlorine and R¹ and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Particularly preferred substituted 1H-quinoxalin-2-one compounds of the general formula I and the tautomers thereof are also those in which R³ denotes a methyl group or a chlorine and R¹, R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Particularly preferred substituted 1H-quinoxalin-2-one compounds of the general formula I and the tautomers thereof are also those in which R¹ and R³ in each case denote a methyl group or a chlorine and R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Preferred substituted 1H-quinoxalin-2-one compounds of the general formula I and the tautomers thereof are furthermore those in which A denotes a bridge of one of the following formulae: —CH₂—, —CH₂—CH₂—, —COO—, —(CH₂)ₙCONH—, wherein n denotes 0, 1 or 2, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

Preferred substituted 1H-quinoxalin-2-one compounds of the general formula I and the tautomers thereof are furthermore those in which X denotes a residue of the following formula

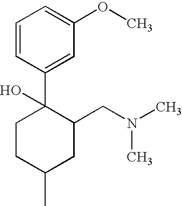

optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

The following substituted 1H-quinoxalin-2-one compounds and the tautomers thereof are very particularly preferred:

6,7-Dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]amide,

6,7-Dichloro-3-oxo-3,4-dihydroquinoxaline 2-carboxylic acid [3′-(N,N-dimethylaminomethyl)-4′-hydroxy-4′-(m-methoxyphenyl)-cyclohexyl]amide,

6,7-Dimethyl-3-oxo-3,4-dihydroquinoxaline 2-carboxylic acid [3′-(N,N-dimethylaminomethyl)-4′-hydroxy-4′-(m-methoxyphenyl)-cyclohexyl] ester,

6,7-Dichloro-3-oxo-3,4-dihydroquinoxaline 2-carboxylic acid [3′-(N,N-dimethylaminomethyl)-4′-hydroxy-4′-(m-methoxyphenyl)-cyclohexyl] ester,

6,7-Dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]propionamide,

6,7-Dichloro-3-oxo-3,4-dihydroquinoxaline 2-carboxylic acid [3′-(N,N-dimethylaminomethyl)-4′-hydroxy-4′-(m-methoxyphenyl)-cyclohexyl]propionamide,

optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

The present invention also provides a process for the production of substituted 1H-quinoxalin-2-one compounds of the above-stated general formula I, the tautomers thereof or corresponding stereoisomers, characterised in that

A) an optionally substituted o-phenylenediamine of the general formula (1), in which R¹, R², R³ and R⁴ have the above-stated meaning, is reacted

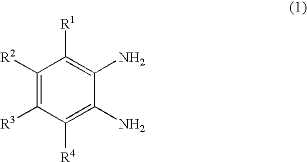

with a 2-ketodicarboxylic acid or a corresponding mono- or dialkyl ester of the general formula (2), in which R denotes a hydrogen or an alkyl group, preferably a methyl group or an ethyl group, and n has the above-stated meaning,

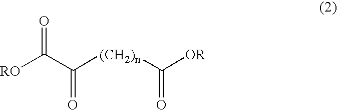

in the presence of an inorganic acid, preferably hydrochloric acid, in a suitable solvent at elevated temperature, preferably at 90-100° C., and is then worked up and the compound formed of the formula Y—COOR, in which R has the above-stated meaning and Y denotes a residue of the general formula Y, in which the unoccupied bond line symbolises the bond to the residue —COOR and

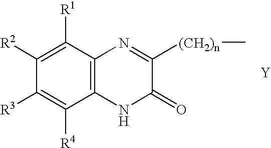

in which R¹, R², R³, R⁴ and n have the above-stated meaning, is optionally purified,

B) an optionally present ester of the formula Y—COOR, in which R denotes an alkyl group, preferably a methyl or ethyl group, is optionally saponified in the presence of a base, preferably sodium or potassium hydroxide, in a suitable solvent, preferably an alcohol/water mixture, particularly preferably in a methanol or ethanol/water mixture, and is then worked up and the carboxylic acid formed of the formula Y—COOH is optionally purified,

C) a carboxylic acid or a carboxylic acid ester of the formula Y—COOR, in which Y has the above-stated meaning and R denotes hydrogen or an alkyl group, preferably a methyl- or ethyl group, is optionally derivatised in that

a) a carboxylic acid or a carboxylic acid ester of the formula Y—COOR is reduced with the assistance of reducing agents, preferably lithium aluminium hydride, in a suitable solvent, preferably tetrahydrofuran, to yield the corresponding alcohol of the formula Y—CH₂—OH,

b) a carboxylic acid or carboxylic acid ester of the formula Y—COOR is reduced with the assistance of reducing agents, preferably diisobutylaluminium hydride, in a suitable solvent, preferably hexane, to yield the corresponding aldehyde of the formula Y—CHO,

c) an alcohol of the formula Y—CH₂—OH according to a) is reacted with a brominating agent, preferably PBr₃ or Ph₃PBr₂ to yield the corresponding bromide of the formula Y—CH₂—Br or

d) a carboxylic acid of the formula Y—COOH, wherein in the above-stated formula Y n denotes O is reacted firstly with (PhO)₂—P(O)—N₃ in a suitable solvent at elevated temperature and then with water to yield the corresponding amine of the formula Y—NH₂

and is then worked up and the product is optionally purified,

D) a compound of the formula X—R′, in which X has the above-stated meaning and R′ denotes a functional group, is optionally derivatised in that

a) a ketone of the formula X═O is reacted 1) with methoxymethyl triphenylphosphinium chloride under protective gas in a suitable solvent, preferably in dimethylformamide, in the presence of sodium hydride and then with hydrochloric acid or 2) with Me₃S⁺BF₄⁻ to yield the corresponding aldehyde X—CHO extended by one carbon atom,

b) an aldehyde of the formula X—CHO according to a) is reacted with a reducing agent, preferably sodium borohydride, in a suitable solvent, preferably an ethanol/water mixture, to yield the corresponding alcohol X—CH₂—OH,

c) an alcohol X—CH₂—OH according to b) or of the formula X—OH is reacted with a brominating agent, preferably triphenylphosphine dibromide, in a suitable solvent, preferably acetonitrile, to yield the corresponding bromide of the formula X—CH₂—Br or X—Br,

d) a bromide of the formula X—CH₂—Br according to c) is reacted with a phosphine of the formula PR″₃, in which R″ denotes an organic residue, preferably a phenyl residue, in a suitable solvent, preferably toluene, ether, tetrahydrofuran or acetone, with cooling and under protective gas to yield the corresponding phosphonium salt R″₃P⁺—CHX⁻,

e) a bromide of the formula X—CH₂—Br according to c) is reacted with a phosphite of the formula HP(O) (OR″′)₂, in which R″′ denotes an organic residue, at elevated temperature, preferably 200° C., to yield the corresponding phosphonate (R″′O)₂P(O)—CH₂—X

and is then worked up and the product is optionally purified,

E) a compound from step A), B) or C), in which Y has the above-stated meaning, is reacted with a compound from step D) or a compound of the formula X—R′, in which X and R′ have the above-stated meaning, in that

a) a carboxylic acid of the formula Y—COOH is reacted with an amine of the formula X—NH₂ in the presence of a suitable condensing agent, preferably dicyclohexyl carbodiimide, 1-hydroxybenzotriazole and N-methylmorphine, in a suitable solvent, preferably dimethylformamide, with formation of an amide bridge,

b) a carboxylic acid of the formula Y—COOH is reacted with an alcohol of the formula X—OH in the presence of a suitable condensing agent in a suitable solvent with formation of an ester bridge, the reaction preferably taking place in the presence of methylimidazole and 1-(mesitylene-2′-sulfonyl)-3-nitro-1,2,4-triazole in tetrahydrofuran or in the presence of dicyclohexylcarbodiimide, 1-hydroxybenzotriazole and N-methylmorphine in dimethylformamide,

c) a bromide of the formula Y—CH₂—Br is reacted with a compound of the formula X—CO(CH₂)ₚ—OH, in which p has the above-stated meaning, under protective gas in the presence of a suitable catalyst, preferably sodium hydride or potassium tert-butylate, in a suitable solvent, preferably dimethylformamide, with formation of a bridge of the formula —CO(CH₂)ₚ—O—CH₂,

d) an alcohol of the formula Y—CH₂—OH is reacted with a bromide of the formula X—Br under protective gas in the presence of a suitable condensing agent, preferably sodium hydride or potassium tert-butylate, in a suitable solvent, preferably dimethylformamide, with formation of an ether bridge,

e) a bromide of the formula Y—CH₂—Br is reacted with an alcohol of the formula X—OH under protective gas in the presence of a suitable condensing agent, preferably sodium hydride or potassium tert-butylate, in a suitable solvent, preferably dimethylformamide, with formation of an ether bridge,

f) an aldehyde of the formula Y—CHO is reacted with an amine of the formula X—NHR^{1′} in the presence of a suitable reducing agent, preferably sodium cyanoborohydride and sodium triacetoxyborohydride, in a suitable solvent, preferably a mixture of tetrahydrofuran and 1,2-dichloroethane, with formation of an amino bridge,

g) an amine of the formula Y—NH₂, wherein in the above-stated formula Y n denotes O is reacted with a bromide of the formula X—(CH₂)ᵣBr in the presence of a suitable catalyst, preferably caesium carbonate, in a suitable solvent, preferably dimethylformamide, with formation of an —NH—(CH₂)ᵣ— bridge,

h) an aldehyde of the formula Y—CHO is reacted with a phosphonium salt R″₃P⁺—CHX⁻, in which R″ has the above-stated meaning, under protective gas in the presence of suitable catalysts in a suitable solvent, preferably in the presence of sodium methanolate in a mixture of hexane, diethyl ether and/or diisopropyl ether or in the presence of sodium hydride, potassium tert-butylate or a lithium amide in dimethylformamide or dimethyl sulfoxide, with formation of a —CH═CH— bridge or

i) an aldehyde of the formula Y—CHO is reacted with a phosphonate of the formula (R″′O)₂P(O)—CH₂—X, in which R″′ has the above-stated meaning, under protective gas in the presence of suitable catalysts, preferably sodium methanolate, sodium hydroxide, potassium hydroxide, sodium hydride, potassium tert-butylate or a lithium amide, in a suitable solvent, preferably dimethylformamide, dimethyl sulfoxide, diethyl ether, tetrahydrofuran, with formation of a —CH═CH— bridge and

j) optionally the —CH═CH— bridge from step h) or i) is hydrogenated by hydrogen, preferably at standard pressure or elevated pressure of up to 100 bar, in the presence of suitable catalysts, preferably transition metals or transition metal compounds, preferably palladium or the salts thereof, rhodium or the complexes thereof, in a suitable solvent, preferably dimethylformamide, methanol or ethanol, at a temperature of between 20 and 100° C. with formation of a —CH₂—CH₂— bridge

and is then worked up and the product is optionally purified.

The solvents and reaction conditions used correspond to the solvents and reaction conditions conventional for these types of reactions.

The starting compounds used for synthesising the 1H-quinoxalin-2-one skeleton, 2-ketodicarboxylic acids of the general formula (2) and optionally substituted o-phenylenediamines of the general formula (1) are commercially obtainable or may be obtained in accordance with conventional methods known to the person skilled in the art.

The reaction of o-phenylenediamines with 2-ketodicarboxylic acids for the synthesis of the 1H-quinoxalin-2-one skeleton is known from E. Campaigne, A. R. McLaughlin, Journal of Heterocyclic Chemistry, 20, 623 (1983); Platt, Sharp, Journal of Chemical Society, 2129, 2133 (1948); Gore, Hughes, Journal of the American Chemical Society, 77, 5738 (1955); V. Colotta, D. Catarzi, F. Varano, L. Cecchi, G. Filacchioni, A. Gallo, G. Costagli, Arch. Pharm. Med. Chem., 330, 129 (1997) and the literature in each case cited therein. Optionally, derivatisation reactions are necessary which introduce the functional groups for linking the 1H-quinoxalin-2-one skeleton to the residue X via the bridge A. The saponification of esters proceeds in accordance with conventional methods known to the person skilled in the art. The other reactions are known from the following literature and literature cited therein: the reduction of carboxylic acids or carboxylic acid esters to yield alcohols from O. Vogl, M. Pöhm, Monatsh. Chem. 83, 541 (1952); A. K. Saund, N. K. Mathur; Ind. J. Chem. 9, 936 (1971), the reduction of carboxylic acids or carboxylic acid esters to yield aldehydes A. Ito, R. Takahashi, Y. Baba; Chem. Pharm. Bull, 23, 3081 (1975); E. Winterfeld; Synthesis (1975), 617; H. Khatri, C. H. Stammer; J. Chem. Soc., Chem. Commun. (1979), 79; D. H. Rieh, E. T. O. Sun; J. Med. Chem. 23, 27 (1980), the reaction of alcohols to yield bromides from J. Am Chem. Soc. 48, 1080 (1926); J. Chem. Soc., 636 (1943); Org. Synth. Coll., Vol. 2, 358 (1943); Liebigs Ann. Chem. 626, 26 (1959); J. Am. Chem. Soc, 86, 964 (1964); J. Am. Chem. Soc. 99, 1612 (1977) and the reaction of carboxylic acids to yield amines from J. Am. Chem. Soc. 94, 6203 (1972), Tetrahedron, 30, 2151 (1974), Org. React. 3, 337 (1947) and Org. Synth. Coll. 5, 273 (1973).

Compounds with residues which are among the general residues X²-X¹⁸, are known from the following literature: X² and X⁵ from German patent application P 3217639, X⁴ from D. Lednicer, J. Med. Chem., 15, 1235 (1972), X³ and X⁶ from German patent application P 19525137, X⁷ and X¹⁰-X¹⁴ from E. Friderichs, T. Christoph, H. Buschmann; Analgesics and Antipyretics; in: J. E. Bailey (ed.); Ullmann's Encyclopedia of Industrial Chemistry, 6th edition, Wiley-VCH, Weinheim and A. F. Casy, R. T. Parfitt; Opioid Analgesics, Plenum Press, New York, X⁸ from Forsyth, J. Chem. Soc., 127, 1666 (1925) and P. A. Grieco, J. Org. Chem., 55, 2271 (1990), X⁹ from Shui, Synth. Commun., 27, 175 (1997), Balsamo, Chim. Ind. (Milan), 58, 519 (1976), Iselin, Helv. Chim. Acta, 37, 178 (1954), X¹⁶ from German patent applications P 101356366 and P 101356374, X¹⁷ from S.-H. Zkao, Tetrahedron Letters, 37, 4463 -(1996); M. Nishiyama, Tetrahedron Letters, 39, 617 (1998); Jain, J. Med. Chem., 10, 812 (1967), X¹⁸ from American patent application U.S. Pat. No. 3,041,344 and van de Westeringh, J. Med. Chem., 7, 619 (1964). X¹⁵ is known as metamizole in the literature and is commercially obtainable.

Compounds X—OH, X—NHR¹, X—CO(CH₂)ₚOH, X—(CH₂)₂—Br and X═O are known from the literature or may be produced from known commercially obtainable compounds in accordance with conventional methods known to the person skilled in the art or in accordance with methods, such as are described in German patent application P100494811.

Derivatisation reactions are optionally required which introduce the functional groups for linking the residue X with the 1H-quinoxalin-2-one skeleton via the bridge A. These reactions may proceed in accordance with conventional methods known to the person skilled in the art and are known from the following literature and the literature cited therein: the reaction of ketones to yield aldehydes extended by one carbon from German patent application P 100494811; J. Nat. Prod., 44, 557 (1981) and Synth. Commun. 12, 613, (1982), the reduction of aldehydes to yield alcohols from German patent application P 100494811 and Chem. Commun. 535 (1975), the reaction of alcohols to yield bromides from J. Am Chem. Soc. 48, 1080 (1926); J. Chem. Soc., 636 (1943); Org. Synth. Coll., Vol. 2, 358 (1943); Liebigs Ann. Chem. 626, 26 (195.9); J. Am. Chem. Soc, 86, 964 (1964); J. Am. Chem. Soc. 99, 1612 (1977), the preparation of phosphonates and phosphonium salts is known from M. Schlosser, Top. Stereochem. 5, 1, (1970); R. Broos, D. Tavernier, M. Anteunis, J. Chem. Educ., 55, 813 (1978); G. Wittig, Angew. Chem. 92, 671 (1980); H. J. Bestmann; Pure Appl. Chem. 52, 771 (1980) and L. Horner, H. Hoffmann, H. G. Wippel, G. Klahre; Chem. Ber. 92, 2499 (1959); J. Gillois, G. Guillerm, M. Savignac, E. Stephan, L. Vo Quang, J. Chem. Educ., 57, 161 (1980); B. A. Arbusov; Pure Appl. Chem. 9, 307 (1964); A. K. Bhattacharya, G. Thyagarajan; Chem. Rev. 81, 415 (1981).

Linkage of the residue X with the 1H-quinoxalin-2-one skeleton via the bridge A may proceed in accordance with conventional methods known to the person skilled in the art and is known from the following literature and the literature in each case cited therein: the reaction of carboxylic acids with alcohols or amines in the presence of dicyclohexylcarbodiimide from W. Konig, R. Geiger, Chem. Ber. 103, 788 (1970), the reaction of carboxylic acids with alcohols in the presence of 1-(mesitylene-2′-sulfonyl)-3-nitro-1,2,4-triazole from Tetrahedron 36, 3075 (1980), etherification from Tetrahedron 35, 2169 (1979), Tetrahedron Lett. (1973), 21; Synthesis, 434 (1974); J. Org. Chem. 52, 4665 (1987), reductive amination from Org. React 3, 174 (1948); J. Am. Chem. Soc. 91, 3996 (1969); Org. Prep. Proced. Int. 11, 201 (1979); Org. Prep. Proced. Int 17, 317 (1985), the Wittig or Wittig-Horner-Emmons reaction from G. Wittig, Angew. Chem. 92, 671 (1980); H. J. Bestmann; Pure Appl. Chem. 52, 771 (1980) and L. Horner, H. Hoffmann, H. G. Wippel, G. Klahre; Chem. Ber., 92, 2499 (1959); J. Gillois, G. Guillerm, M. Savignac, E. Stephan, L. Vo Quang; J. Chem. Educ. 57, 161 (1980); B. A. Arbusov; Pure Appl. Chem. 9, 307 (1964); A. K. Bhattacharya, G. Thyagarajan; Chem. Rev. 81, 415 (1981) and hydrogenation from Synthesis (1978), 329; J. Org. Chem. 34, 3684 (1969); J. Am. Chem. Soc. 91, 2579 (1969).

The corresponding literature descriptions are hereby introduced as a reference and are deemed to be part of the disclosure.

The substituted 1H-quinoxalin-2-one compounds of the general formula I according to the invention and the above-excepted compounds, the tautomers thereof and in each case corresponding stereoisomers may be isolated both in the form of the free bases thereof and in the form of corresponding salts.

The free bases of the respective compounds according to the invention of the general formula I and of the above-excepted compounds, the tautomers and respective corresponding stereoisomers thereof may be converted into the corresponding physiologically acceptable salts by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid. The free bases of the respective compounds according to the invention of the general formula I and of the above-excepted compounds, the tautomers and respective corresponding stereoisomers thereof may preferably be converted into the corresponding hydrochlorides by combining the compounds according to the invention of the general formula I or the above-excepted compounds, the tautomers or corresponding stereoisomers thereof as free bases, dissolved in a suitable organic solvent, such as for example butane-2-one (methyl ethyl ketone), with trimethylsilyl chloride (TMSCl).

The free bases of the respective compounds according to the invention of the general formula I and of the above-excepted compounds, the tautomers and respective corresponding stereoisomers thereof may be converted into the corresponding physiologically acceptable salts with the free acid or a salt of a sugar substitute, such as for example saccharin, cyclamate or acesulfame.

The compounds according to the invention of the general formula I and the above-excepted compounds, the tautomers and respective corresponding stereoisomers thereof may optionally, like the corresponding acids, the corresponding bases or salts of these compounds, also be obtained in the form of the solvates thereof, preferably the hydrates thereof.

If the substituted 1H-quinoxalin-2-one compounds according to the invention of the general formula I, the above-excepted compounds or the tautomers thereof are obtained by the production process according to the invention in the form of stereoisomers, preferably in the form of the racemates thereof or other mixtures of their various enantiomers and/or diastereomers, these may be separated and optionally isolated by conventional processes known to the person skilled in the art. Examples which may be mentioned are chromatographic separation processes, in particular liquid chromatography processes at standard pressure or at elevated pressure, preferably MPLC and HPLC processes, and fractional crystallisation processes. Individual enantiomers, e.g. diastereomeric salts formed by means of HPLC on a chiral phase or by means of crystallisation with chiral acids, such as (+)-tartaric acid, (−)-tartaric acid or (+)-10-camphorsulfonic acid, may here in particular be separated from one another.

The substituted 1H-quinoxalin-2-one compounds according to the invention of the general formula I and substituted 1H-quinoxalin-2-one compounds of the general formula I, in which the residue X⁷ is attached via an amide bridge, respective tautomers thereof and corresponding stereoisomers as well as in each case the corresponding, bases, salts and solvates are toxicologically safe and are therefore suitable as pharmaceutical active ingredients in pharmaceutical preparations.

The present invention accordingly further provides pharmaceutical preparations, which contain at least one substituted 1H-quinoxalin-2-one compound according to the invention of the general formula I and/or the tautomer thereof and/or at least one substituted 1H-quinoxalin-2-one compound of the general formula I and/or the tautomer thereof in which the residue X⁷ is attached via an amide bridge, optionally in each case in the form of the racemate thereof, the pure stereoisomer thereof, in particular enantiomer or diastereomer, or in the form of mixtures of the stereoisomers, in particular the enantiomers or diastereomers, in any desired mixing ratio or in each case in the form of the acid or base thereof or in the form of the salt thereof, in particular a physiologically acceptable salt, or in the form of the solvate thereof, in particular the hydrate, optionally together with physiologically acceptable auxiliary substances. It goes without saying that the pharmaceutical preparations according to the invention may also contain mixtures of two or more of the above-stated compounds.

If the substituted 1H-quinoxalin-2-one compounds according to the invention of the general formula I or the corresponding compounds in which the residue X⁷ is attached via an amide bridge or the tautomers thereof or the corresponding bases, salts or solvates thereof are chiral, they may be present in the pharmaceutical preparation according to the invention, as already stated, preferably in the form of the racemates thereof, the pure enantiomers thereof, the pure diastereomers thereof, or in the form of a mixture of at least two of the above-stated stereoisomers.

The pharmaceutical preparations according to the invention are preferably suitable for the treatment or prevention of cerebral oedema, psychoses brought about by elevated amino acid levels, AIDS dementia, Tourette's syndrome, encephalomyelitis, tinnitus, migraine, inflammatory and/or allergic reactions, depression, mental health conditions, urinary incontinence, pruritus, diarrhoea or for anxiolysis.

The present invention accordingly further provides pharmaceutical preparations, which contain at least one substituted 1H-quinoxalin-2-one compound according to the invention of the general formula I or the tautomer thereof, optionally in the form of the racemate thereof, the pure stereoisomer thereof, in particular enantiomer or diastereomer, or in the form of mixtures of the stereoisomers, in particular the enantiomers or diastereomers, in any desired mixing ratio or in each case in the form of the acid or base thereof or in the form of the salt thereof, in particular a physiologically acceptable salt, or in the form of the solvate thereof, in particular the hydrate, optionally together with physiologically acceptable auxiliary substances. It goes without saying that the pharmaceutical preparations according to the invention may also contain mixtures of two or more of the above-stated compounds.

If the substituted 1H-quinoxalin-2-one compounds according to the invention of the general formula I and the tautomers thereof or the corresponding bases, salts or solvates thereof are chiral, they may be present in the pharmaceutical preparation according to the invention, as already stated, preferably in the form of the racemates thereof, the pure enantiomers thereof, the pure diastereomers thereof, or in the form of a mixture of at least two of the above-stated stereoisomers.

These pharmaceutical preparations according to the invention are preferably suitable for combatting pain, preferably chronic or neuropathic pain, or for the treatment or prevention of stroke, neurodegenerative diseases, preferably Alzheimer's disease, Parkinson's disease, Huntington's chorea, or for the treatment or prevention of cerebral infarct, cerebral ischaemia, insufficiency states of the central nervous system, preferably hypoxia or anoxia, epilepsy, schizophrenia, perinatal asphyxia or for anaesthesia.

The present invention also provides the use of at least one substituted 1H-quinoxalin-2-one compound according to the invention of the general formula I and/or the tautomers thereof and/or at least one substituted 1H-quinoxalin-2-one compound of the general formula I and/or the tautomers thereof in which the residue X⁷ is attached via an amide bridge, in each case optionally in the form of the racemate thereof, the pure stereoisomer thereof, in particular enantiomer or diastereomer, or in the form of mixtures of the stereoisomers, in particular the enantiomers or diastereomers, in any desired mixing ratio, or in each case in form of the acid or base thereof or in the form of the salt thereof, in particular a physiologically acceptable salt, or in the form of the solvate thereof, in particular the hydrate, for the production of a pharmaceutical preparation for the treatment or prevention of stroke, cerebral oedema, psychoses brought about by elevated amino acid levels, AIDS dementia, Tourette's syndrome, encephalomyelitis, tinnitus, migraine, inflammatory and/or allergic reactions, depression, mental health conditions, urinary incontinence, pruritus, diarrhoea or for anxiolysis.

The present invention further provides the use of at least one substituted 1H-quinoxalin-2-one compound according to the invention of the general formula I or the tautomers thereof, optionally in the form of the racemate thereof, the pure stereoisomer thereof, in particular enantiomer or diastereomer, or in the form of mixtures of the stereoisomers, in particular of the enantiomers or diastereomers, in any desired mixing ratio, or in each case in the form of the acid or base thereof or in the form of the salt thereof, in particular of a physiologically acceptable salt, or in the form of the solvate thereof, in particular the hydrate, for the production of a pharmaceutical preparation for combatting pain, preferably chronic or neuropathic pain, and for the treatment or prevention of neurodegenerative diseases, preferably Alzheimer's disease, Parkinson's disease or Huntington's chorea, cerebral infarct, cerebral ischaemia, insufficiency states of the central nervous system, preferably hypoxia or anoxia, epilepsy, schizophrenia, perinatal asphyxia or for anaesthesia.

The pharmaceutical preparations according to the invention may be present as liquid, semisolid or solid dosage forms, for example in the form of solutions for injection, drops, succi, syrups, sprays, suspensions, tablets, patches, capsules, transdermal delivery systems, suppositories, ointments, creams, lotions, gels, emulsions, aerosols or in multiparticulate form, for example in the form of pellets or granules, and also be administered as such.

In addition to at least one substituted 1H-quinoxalin-2-one compound according to the invention of the general formula I and/or at least one substituted 1H-quinoxalin-2-one compound of the general formula I in which the residue X⁷ is attached via an amide bridge, or the tautomer thereof, optionally in the form of the racemate thereof, the pure stereoisomer thereof, in particular enantiomer or diastereomer, or in the form of mixtures of the stereoisomers, in particular the enantiomers or diastereomers, in any desired mixing ratio, or in each case in form of the acid or base thereof or in the form of the salt thereof, in particular a physiologically acceptable salt, or in the form of the solvate thereof, in particular the hydrate, the pharmaceutical preparations according to the invention conventionally contain further physiologically acceptable pharmaceutical auxiliary substances, which are preferably selected from the group consisting of matrix materials, fillers, solvents, diluents, surface-active substances, dyes, preservatives, suspending agents, slip agents, lubricants, aromas and binders.

Selection of the physiologically acceptable auxiliary substances and the quantities thereof which are to be used depends upon whether the pharmaceutical preparation is to be administered orally, subcutaneously, parenterally, intravenously, intraperitoneally, intradermally, intramuscularly, intranasally, buccally, rectally or topically, for example onto infections of the skin, mucous membranes or eyes. Preparations in the form of tablets, coated tablets, capsules, granules, pellets, drops, succi and syrups are preferred for oral administration, while solutions, suspensions, readily reconstitutible dried preparations and sprays are preferred for parenteral, topical and inhalatory administration.

Compounds according to the invention of the general formula I or a substituted 1H-quinoxalin-2-one compound of the general formula I in which the residue X⁷ is attached via an amide bridge, or the tautomers thereof, optionally in the form of the racemate thereof, the pure stereoisomer thereof, in particular enantiomer or diastereomer, or in the form of mixtures of the stereoisomers, in particular the enantiomers or diastereomers, in any desired mixing ratio, or in each case in form of the acid or base thereof or in the form of the salt thereof, in particular a physiologically acceptable salt, or in the form of the solvate thereof, in particular the hydrate, in a depot in dissolved form or in a dressing, optionally with the addition of skin penetration promoters, are suitable percutaneous administration preparations. Orally or percutaneously administrable formulations may also release the corresponding compounds in delayed manner.

Production of the pharmaceutical preparations according to the invention proceeds with the assistance of conventional means, devices, methods and processes known to the person skilled in the art, such as are described for example in “Remington's Pharmaceutical Sciences”, ed. A. R. Gennaro, 17th ed., Mack Publishing Company, Easton, Pa. (1985), in particular in part 8, chapters 76 to 93. The corresponding literature description is hereby introduced as a reference and is deemed to be part of the disclosure. The quantity of the particular substituted 1H-quinoxalin-2-one compound according to the invention of the general formula I or of the substituted 1H-quinoxalin-2-one compound of the general formula I in which the residue X⁷ is attached via an amide bridge, the tautomer thereof, optionally in the form of the racemate thereof, the pure stereoisomer thereof, in particular enantiomer or diastereomer, or in the form of mixtures of the stereoisomers, in particular the enantiomers or diastereomers, in any desired mixing ratio, or in each case in form of the acid or base thereof or in the form of the salt thereof, in particular a physiologically acceptable salt, or in the form of the solvate thereof, in particular the hydrate, to be administered to the patient may vary and is for example dependent on the weight or age of the patient and on the mode of administration, the indication and the severity of the complaint. Conventionally, at least one corresponding compound is administered in a quantity of 0.005.to 500 mg/kg, preferably of 0.05 to 5 mg/kg, of patient body weight.

The present invention also provides substituted 4-aryl- and 4-heteroarylcyclohexane compounds of the general formula II,

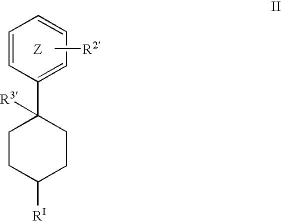

in which

R^{I} denotes a keto or aldehyde group or a group of the formula —NHR¹, —CO—(CH₂)ₚ—OH, —(CH₂)ᵣOH or —(CH₂)ᵣBr, wherein R^{1′} has the meaning stated hereinafter and p denotes 0 or 1 and r denotes 0, 1 or 2,

R^{1′} denotes hydrogen, a linear or branched, saturated or unsaturated aliphatic C₁₋₁₀ residue, a saturated or unsaturated cycloaliphatic C₃₋₇ residue, an aryl or heteroaryl residue,

R^{2′} denotes a linear or branched, saturated or unsaturated aliphatic C₁₋₁₀ residue, a saturated or unsaturated cycloaliphatic C₃₋₇ residue or an aryl- or heteroaryl residue, wherein all the above-stated residues may optionally be joined via an ether, thioether or SO₂ bridge, or hydrogen, a halogen, a hydroxy, thiol, cyano or nitro group or a group of the formula —CH₂F, —CHF₂, —CF₃ or —NR^{1′}₂, wherein the two residues R^{1′} are identical or different and have the above-stated meaning,

R^{3′} denotes a linear or branched, saturated or unsaturated aliphatic C₁₋₁₀ residue, a saturated or unsaturated cycloaliphatic C₃₋₇ residue, an aryl or heteroaryl residue, wherein all the above-stated residues may optionally be joined via an ether or an ester bridge, hydrogen, a halogen, a hydroxy group and

Z denotes at least one optionally present oxygen, sulfur or nitrogen as a ring atom,

optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates,

with the exception of cis-4-amino-1-phenylcyclohexanol, trans-4-ethanoyl-1-phenylcyclohexane, trans-4-butanoyl-1-phenylcyclohexane and compounds of the general formula IIa,

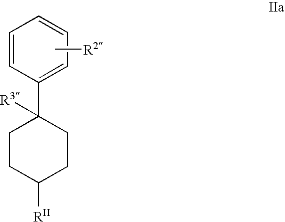

in which

R^{II} denotes a phenyl or naphthyl residue attached via an NH bridge,

R^{2′} denotes hydrogen, a lower alkoxy residue, an amino or a nitro group and

R^{3′} denotes hydrogen or a hydroxy group.

Preferred substituted 4-aryl- and 4-heteroarylcyclohexane compounds of the formula X¹—R^{I} are those which are characterised in that R^{I} denotes a keto, hydroxy or amino group, R^{2′} denotes a hydroxy group or alkoxy group with a linear or branched, saturated or unsaturated aliphatic C₁₋₃ residue and R^{3′} denotes a hydroxy group, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

The following substituted 4-aryl- and 4-heteroarylcyclohexane compounds are very particularly preferred:

4-Hydroxy-4-(3′-methoxyphenyl)cyclohexan-1-one,

4-Hydroxy-4-(3′-methoxyphenyl)cyclohexan-1-ol,

4-Amino-4-(3′-methoxyphenyl)cyclohexan-1-ol and

4-Amino-4-(3′-hydroxyphenyl)cyclohexan-1-ol,

optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

The present invention also provides a process for the production of substituted 4-aryl- and 4-heteroarylcyclohexane compounds of the general formula II or corresponding stereoisomers, in which

a) 1,4-cyclohexanedione monoethylene ketal, 4-aminocyclohexan-1-one ethylene ketal or 4-oxocyclohexanecarboxylic acid is reacted with magnesium and a brominated or chlorinated, optionally substituted aromatic or heteroaromatic compound in a suitable solvent, preferably dry diethyl ether, at elevated temperature to yield the corresponding coupling product and then the ketal is optionally cleaved by reaction with hydrochloric acid in a suitable solvent, preferably tetrahydrofuran, and worked up, optionally followed by purification of the product of the formula X^{1a}═O, X^{1a}—NHRR^{1′} or X^{1a}—CO₂H, in which X^{1a} denotes a residue of the formula X^{1a} and R^{′}, R^{2′} and Z have the above-stated meaning and the unoccupied bond line symbolises the bond to the respective residue ═O, —NHR^{1′} or —CO₂H,

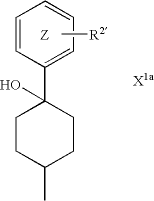

b) a ketone of the formula X^{1a}═O is optionally reacted in the presence of a suitable reducing agent, preferably sodium borohydride, in a suitable solvent, preferably methanol, to yield the corresponding alcohol of the formula X^{1a}—OH, is worked up and the product is optionally purified,

c) a ketone of the formula X^{1a}═O is optionally reacted under nitrogen in a suitable solvent, preferably tetrahydrofuran, firstly with ammonium trifluoroacetate and then with glacial acetic acid and sodium triacetoxyborohydride, to yield the corresponding amine of the formula X^{1a}—NH₂, is worked up and the product is optionally purified,

d) a carboxylic acid of the formula X^{1a}—CO₂H is optionally activated by reaction with dicyclohexylcarbodiimide or by conversion into the carboxylic acid chloride or a mixed anhydride, is reacted with diazomethane in a suitable solvent, preferably ether, and is then treated with water, worked up and the product of the formula X^{1a}—CO—CH₂—OH is optionally purified, e) a compound from step d) is optionally reacted firstly in the presence of a suitable reducing agent in a suitable solvent to yield a compound of the formula X^{1a}—(CH₂)₂—OH and then this compound is reacted with a brominating agent, preferably PPh₃/Br₂, in a suitable solvent to yield a compound of the formula X^{1a}—(CH₂)₂—Br, is worked up and the product is optionally purified,

f) a ketone of the formula X^{1a}═O according to a) is reacted 1) with methoxymethyl triphenylphosphinium chloride under protective gas in a suitable solvent, preferably in dimethylformamide, in the presence of sodium hydride and then with hydrochloric acid or 2) with Me₃S⁺BF₄⁻ to yield the corresponding aldehyde X^{1a}—CHO extended by one carbon atom, is then worked up and the product is optionally purified,

g) an aldehyde of the formula X^{1a}—CHO according to f) is reacted with a reducing agent, preferably sodium borohydride, in a suitable solvent, preferably an ethanol/water mixture, to yield the corresponding alcohol X^{1a}—CH₂—OH, is then worked up and the product is optionally purified,

h) an alcohol of the formula X^{1a}—CH₂—OH according to g) or of the formula X^{1a}—OH according to b) is reacted with a brominating agent, preferably triphenylphosphine dibromide, in a suitable solvent, preferably acetonitrile, to yield the corresponding bromide of the formula X^{1a}—CH₂—Br or X^{1a}—Br respectively, is then worked up and the product is optionally purified,

i) the hydroxy group in position 4 of the cyclohexane ring in the residue X^{1a} is optionally converted into hydrogen, a halogen, an ether, ester, aryl or heteroaryl group or into an aliphatic or cycloaliphatic residue, in that

α) in order to introduce an ether group, a compound from one of steps a)-h) is reacted with an aliphatic or cycloaliphatic compound in the presence of a suitable catalyst in a suitable solvent, preferably in the presence of sodium hydride in dimethylformamide or in the presence of potassium hydroxide in dimethyl sulfoxide, or with an alkylating agent in a suitable solvent, preferably with a diazo compound in diethyl ether, or with an aryl or heteroaryl compound in the presence of diethylazo dicarboxylate and triphenylphosphine,

β) in order to introduce a halogen, a compound from one of steps a)-h) is reacted with a halogenating agent in a suitable solvent, preferably with POCl₃ in dimethylformamide, with PPh₃/Cl₂, with PPh₃/Br₂, with triphenylphosphine/n-chlorosuccinimide or with HCl/ZnCl₂,

γ) in order to introduce hydrogen, a compound from step β) is reacted with hydrogen in the presence of a suitable catalyst, preferably palladium/carbon, in a suitable solvent,

δ) in order to introduce an aliphatic or cycloaliphatic residue, or aryl or heteroaryl group, a compound from step β) is reacted with an aliphatic or cycloaliphatic boronic acid or a boronic acid ester or an aryl or heteroaryl borodihydroxide compound in the presence of palladium(II) acetate and potassium carbonate in a suitable solvent, preferably a dimethylformamide/water mixture, or

ε) in order to introduce an ester group, a compound from one of steps a)-h) is reacted with a carboxylic acid chloride in the presence of a suitable catalyst in a suitable solvent

and is then worked up, optionally followed by purification of the compound formed of the formula X¹—R′, in which X¹ denotes the formula X¹

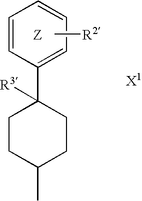

and R^{I}, R^{2′} and R^{3′} have the above-stated meaning.

The starting compounds for the synthesis of compounds with the residue X¹, 1,4-cyclohexanedione monoethylene ketal, 4-oxocyclohexanecarboxylic acid and 4-aminocyclohexan-1-one ethylene ketal are known. 1,4-Cyclohexanedione monoethylene ketal and 4-oxocyclohexanecarboxylic acid are commercially obtainable or may be obtained using conventional methods known to the person skilled in the art. 4-Aminocyclohexan-1-one ethylene ketal is known from H.-J. Teuber, Liebigs Ann. Chem., 781 (1990) and M. Mimura, Chem. Pharm. Bull., 41, 1971 (1993).

The reactions for synthesising compounds X¹-R^{I} proceed according to conventional methods known to the person skilled in the art. The reaction of a cyclohexanone with a chlorinated or brominated, optionally substituted aromatic or heteroaromatic compound is known from Chem. Ber. 68, 1068 (1935), An. Quim. 64, 607 (1968) and Indian J. Biochem. 5, 79 (1968).

The functional group R^{I} is optionally derivatised. These reactions may proceed using conventional methods known to the person skilled in the art and are known from the following literature and the literature cited therein: the reaction of ketones to yield aldehydes extended by one carbon are known from German patent application P 100494811; J. Nat. Prod., 44, 557 (1981) and Synth. Commun, 12, 613 (1982), the reduction of aldehydes to alcohols from German patent application P 100494811 and Chem. Commun. 535 (1975), the reaction of alcohols to yield bromides from J. Am. Chem. Soc. 48, 1080 (1926); J. Chem. Soc., 636 (1943); Org. Synth. Coll, Vol. 2, 358 (1943); Liebigs Ann. Chem. 626, 26 (1959); J. Am. Chem. Soc. 86, 964 (1964); J. Am. Chem. Soc. 99, 1612 (1977).

A modification or exchange of the hydroxy group in position 4 of the cyclohexane ring optionally takes place in the residue X¹. The reactions may be performed in accordance with conventional methods known to the person skilled in the art and are known from the following literature and the literature cited therein: alkylation of the hydroxy group from R. M. Bowman et al, Journal of the Chemical Society (C), 2368 (967); C. G. Neville et al, Journal of the Chemical Society, Perkin Trans. I, 259 (1991); F. Arnt et al, Chemische Berichte, 86, 951 (1953), Journal of Organic Chemistry, 52, 4665 (1987) and Tetrahedron 35, 2169 (1979), arylation or heteroarylation of the hydroxy group from Journal of the American Chemical Society, 107, 3891 (1985), the introduction of a halogen from Journal of the American Chemical Society, 76, 6073 (1954) and Journal of the American Chemical Society, 86, 964 (1964), Journal of the Chemical Society, 636 (1943), Journal of the American Chemical Society, 106, 3286 (1984), Journal of the Chemical Society, 2281 (1954) and Synthesis, 746 (1980), the introduction of an alkyl, aryl or heteroaryl residue from A. Suzuki, Acc. Chem. Res., 15, 178 (1982); A. Suzuki, Pure Appl. Chem., 57, 1749 (1985); A. Suzuki, Pure Appl. Chem., 63, 419 (1991), A. Suzuki, Pure Appl. Chem., 66, 213 (1994), the conversion of chlorides into alkanes from Journal of Organic Chemistry, 23, 1938 (1958), the esterification of the hydroxy group from W. König, R. Geiger, Chem. Ber. 103, 788 (1970).

The investigation into analgesic efficacy was performed by phenylquinone-induced writhing in mice (modified after: I. C. Hendershot, J. Forsaith, J. Pharmacol. Exp. There. 125, 237-240 (1959)). The corresponding literature description is hereby introduced as a reference and is deemed to be part of the disclosure.

Male NMRI mice weighing from 25 to 30 g were used for this purpose. Groups of 10 animals per substance dose received, 10 minutes after intravenous administration of the compounds tested, 0.3 ml/mouse of a 0.02% aqueous solution of phenylquinone (phenylbenzoquinone, Sigma, Deisenhofen; solution prepared with addition of 5% of ethanol and stored in a water bath at 45° C.) administered intraperitoneally. The animals were placed individually in observation cages. A push button counter was used to record the number of pain-induced stretching movements (writhing reactions=straightening of the torso with stretching of the rear extremities) for 5-20 minutes after phenylquinone administration. The control was provided by animals which received only physiological common salt solution.

The compounds were tested at the standard dosage of 10 mg/kg. Inhibition of the writhing reactions by a substance was calculated according to the following formula:

$$\text{\% Inhibition} = 100 - [ \frac{\text{Writhing reaction, treated animals}}{\text{Writhing reaction, control}} \times 100 ]$$

The invention is explained below with reference to Examples. These explanations are given merely by way of example and do not restrict the general concept of the invention.

### EXAMPLE

The yields of the example compounds according to the invention were not optimised.

### Example 1

### Synthesis of 6,7-dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]amide hydrochloride

1st Step:

Preparation of 6,7-dimethyl-3-oxo-3,4-dihydroquinoxaline 2-carboxylic acid ethyl ester

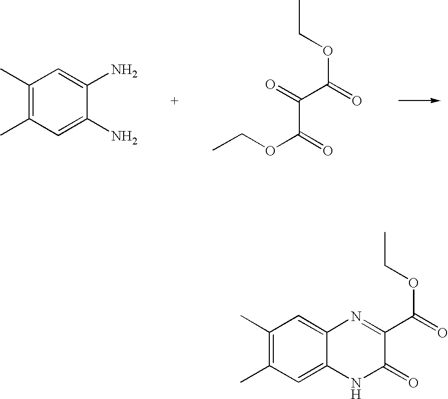

6.81 g (50 mmol) of 1,2-diamino-4,5-dimethylbenzene were dissolved in 180 ml of 2N HCl and combined with 8.7 g (50 mmol) of mesoxalic acid diethyl ester. After 3 h at 100° C., the batch was stirred for a further 12 h at 20° C. The resultant precipitate was removed by suction filtration, washed with water and diethyl ether and dried. Yield of the crude product 6,7-dimethyl-3-oxo-3,4-dihydroquinoxaline 2-carboxylic acid ethyl ester was 7.9 g (32 mmol).

2nd Step

Preparation of 6,7-dimethyl-3-oxo-3,4-dihydroquinoxaline 2-carboxylic acid

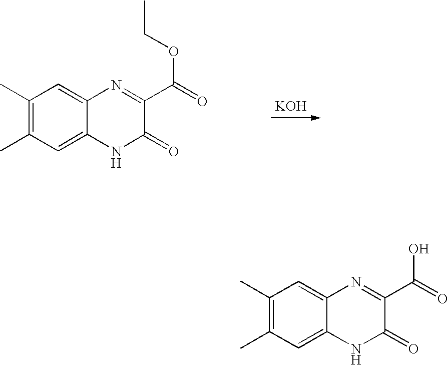

7.9 g (32 mmol) of 6,7-dimethyl-3-oxo-3,4-dihydroquinoxaline 2-carboxylic acid ethyl ester were stirred with 4.5 g (80 mmol) of potassium hydroxide in 60 ml of ethanol and 50 ml of water for 18 h at 20° C. The mixture was then acidified with 2N HCl and the precipitate washed with water. The yield of 6,7-dimethyl-3-oxo-3,4-dihydroquinoxaline 2-carboxylic acid was 6.87 g (98%). The compound had a melting point of 276-285° C.

3rd Step

Preparation of 6,7-dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]amide hydrochloride

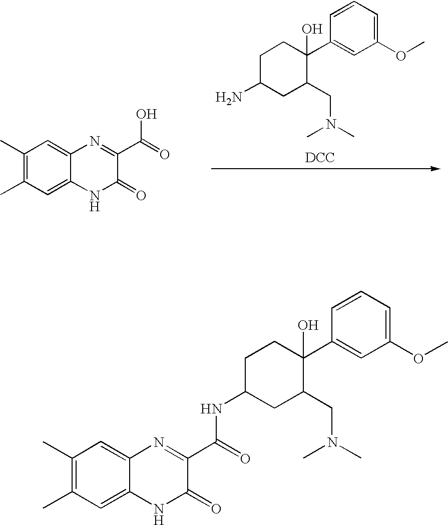

1.0 g (4.58 mmol) of 6,7-dimethyl-3-oxo-3,4-dihydroquinoxaline 2-carboxylic acid were reacted in 60 ml of DMF with 1.27 g (4.58 mmol) of 4-amino-2-(N,N-dimethylaminomethyl)-1-(m-methoxyphenyl)-cyclohexan-1-ol in the presence of 1.89 g (9.16 mmol) of dicyclohexylcarbodiimide (DCC), 1.24 g (9.16 mmol) of 1-hydroxybenzotriazole (HOBT) and 1.0 ml (9.16 mmol) of N-methylmorpholine at 0° C. After two hours, the reaction solution was heated to 20° C. and stirred for 96 h. After separation of the secondary products, the product 6,7-dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]amide was extracted with ethyl acetate from the reaction solution, which had been combined with water and alkalised. Purification was performed by precipitation as the hydrochloride in methyl ethyl ketone with trimethylsilyl chloride. The yield was 214 mg (69%). The melting range of the compound was 245-250° C.

### Example 2

### Synthesis of 6,7-dichloro-3-oxo-3,4-dihydroquinoxaline 2-carboxylic acid [3′-(N,N-dimethylaminomethyl)-4′-hydroxy-4′-(m-methoxyphenyl)-cyclohexyl]amide

6,7-Dichloro-3-oxo-3,4-dihydroquinoxaline 2-carboxylic acid was prepared in a manner similar to Example 1. 259 mg (1 mmol) of this carboxylic acid were reacted with 278.4 mg (1 mmol) of 4-amino-2-(N,N-dimethylaminomethyl)-1-(m-methoxyphenyl)-cyclohexan-1-ol in the presence of dicyclohexylcarbodiimide (DCC), 1-hydroxybenzotriazole (HOBT) and N-methylmorpholine with a yield of 203 mg (48%) to yield 6,7-dichloro-3-oxo-3,4-dihydroquinoxaline 2-carboxylic acid [3′-(N,N-dimethylaminomethyl)-4′-hydroxy-4′-(m-methoxyphenyl)-cyclohexyl]amide. The melting range of the compound was 270-273° C.

### Example 3

### Synthesis of 6,7-dimethyl-3-oxo-3,4-dihydroquinoxaline 2-carboxylic acid [3′-(N,N-dimethylaminomethyl)-4′-hydroxy-4′-(m-methoxyphenyl)-cyclohexyl] ester

6,7-Dimethyl-3-oxo-3,4-dihydroquinoxaline 2-carboxylic acid was prepared in a manner similar to Example 1. 1.0 g (4.58 mmol) of this carboxylic acid was suspended with 272 μl (3.44 mmol) of 1-methylimidazole in 30 ml of THF. 1.27 g (4.58 mmol) of 2-(N,N-dimethylaminomethyl)-1-(m-methoxyphenyl)-cyclohexane-1,4-diol and 1.35 g of 1-(mesitylene-2′-sulfonyl)-3-nitro-1,2,4-triazole were separately dissolved in 25 ml of THF. The mixtures were combined and stirred for 72 h at 20° C. The precipitate was removed by suction filtration and washed with ether and THF. The product was obtained in a yield of 281 mg (43%). The melting range of the compound was 114-118° C.

### Example 4

### Synthesis of 6,7-dichloro-3-oxo-3,4-dihydroquinoxaline 2-carboxylic acid [3′-(N,N-dimethylaminomethyl)-4′-hydroxy-4′-(m-methoxyphenyl)-cyclohexyl] ester

6,7-Dichloro-3-oxo-3,4-dihydroquinoxaline 2-carboxylic acid was prepared in a manner similar to Example 1. 388.5 mg (1.5 mmol) of the acid were dissolved in 30 ml of dry dichloromethane and combined in succession with 444.6 mg (1.5 mmol) of 1-(mesitylene-2′-sulfonyl)-3-nitro-1,2,4-triazole (MSNT), 92.4 mg (1.125 mmol) of 1-methylimidazole and-416.1 mg (1.5 mmol) of 2-(N,N-dimethylaminomethyl)-1-(m-methoxyphenyl)-cyclohexane-1,4-diol. The batch was stirred for 72 h at room temperature, the precipitated solid removed by suction filtration and washed with dichloromethane. In order to eliminate any unreacted MSNT, the mixture was stirred for 1 h with dichloromethane at room temperature. In order to separate a nonpolar secondary product, the solid was stirred with a mixture of acetone/ethyl methyl ketone (1:1) at 55° C. for 30 min. The yield of 6,7-dichloro-3-oxo-3,4-dihydroquinoxaline 2-carboxylic acid [3′-(N,N-dimethylaminomethyl)-4′-hydroxy-4′-(m-methoxyphenyl)-cyclohexyl] ester was 820 mg (35%). The purified product melted at 175-177° C.

### Example 5

### Synthesis of 6,7-dichloro-3-oxo-3,4-dihydroquinoxaline 2-carboxylic acid [3′-(N,N-dimethylaminomethyl)-4′-hydroxy-4′-(m-methoxyphenyl)-cyclohexyl]propionamide

Step 1

Preparation of 3′-(6,7-dichloro-3-oxo-3,4-dihydroquinoxalin-2-yl)propanoic acid

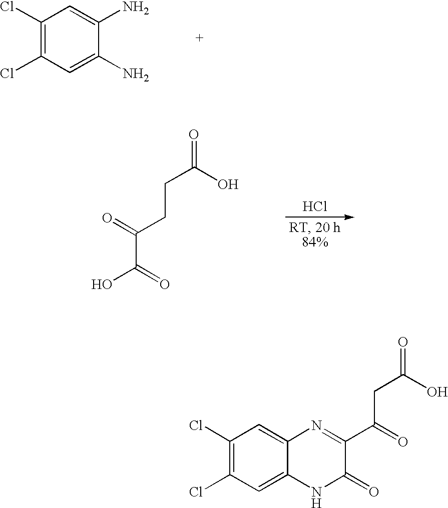

For the preparation of 3′-(6,7-dichloro-3-oxo-3,4-dihydroquinoxalin-2-yl)propanoic acid, 8.85 g (50 mmol) of 1,2-diamino-4,5-dichlorobenzene were suspended and partially dissolved in 180 ml (20-fold quantity) of 2N HCl. 7.3 g (50 mmol) of 2-oxoglutaric acid were added in portions. The suspension was stirred at room temperature. Within 1 h, the brown mixture turned light pink and a light coloured solid precipitated out together with the still visible 1,2-diamino-4,5-dichlorobenzene. After 2 h at room temperature, only traces of the starting materials were still detectable by TLC. Working up was performed by removing the precipitate by suction filtration, washing it thoroughly on the sintered-glass filter with 2N HCl, water and ether and then drying it. The crude product war still contaminated (nonpolar spot in TLC). Purification could be achieved by recrystallisation from acetone. The yield was 11.99 mg (84%). The purified 3′-(6,7-dichloro-3-oxo-3,4-dihydroquinoxalin-2-yl)propanoic acid melted at 286-289° C. and was white.

2nd Step:

Preparation of 3′-(6,7-dichloro-3-oxo-3,4-dihydroquinoxalin-2-yl)propanoic acid-[3′-(N,N-dimethylaminomethyl)-4′-hydroxy-4′-(m-methoxyphenyl)-cyclohexyl]amide

287.1 mg (1 mmol) of 3′-(6,7-dichloro-3-oxo-3,4-dihydroquinoxalin-2-yl)propanoic acid were dissolved in 5 ml of dry DMF and 270.3 mg (2 mmol) of 1-hydroxybenzotriazole, 278.4 mg (1 mmol) of 4-amino-2-(N,N-dimethylaminomethyl)-1-(m-methoxyphenyl)-cyclohexanol and 0.22 ml (2 mmol) of N-methylmorpholine. The clear reaction mixture was cooled to 0° C. and then 412 mg (2 mmol) of dicyclohexylcarbodiimide were stirred in. A precipitate formed as the mixture rose to room temperature. The reaction mixture turned slowly black-grey and the precipitate slowly increased. After 4 days, the batch was worked up. To this end, the reaction mixture was cooled in the refrigerator for 2 h, the solid (dicyclohexylurea) removed by suction filtration and washed with cold DMF. The yield was 321.0 mg (58%). The purified 3″-(6,7-dichloro-3-oxo-3,4-dihydroquinoxalin-2-yl]propanoic acid-[3′-(N,N-dimethylaminomethyl)-4′-hydroxy-4′-(m-methoxyphenyl)cyclohexyl]amide melted at 204-207° C. and was beige-brown.

### Example 6

### Synthesis of 6,7-dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]propionamide

The preparation proceeded in a manner similar to Example 5. Instead of 3′-(6,7-dimethyl-3-oxo-3,4-dihydroquinoxalin-2-yl)propanoic acid, 3′-(6,7-dichloro-3-oxo-3,4-dihydroquinoxalin-2-yl)propanoic acid was used, which was produced in accordance with the same method as in Example 5 from 1,2-diamino-4,5-dimethylbenzene and 2-oxoglutaric acid. The compound had a melting range of 188-192° C.

### Example 7

### Synthesis of 4-hydroxy-4-(3′-methoxyphenyl)cyclohexan-1-one

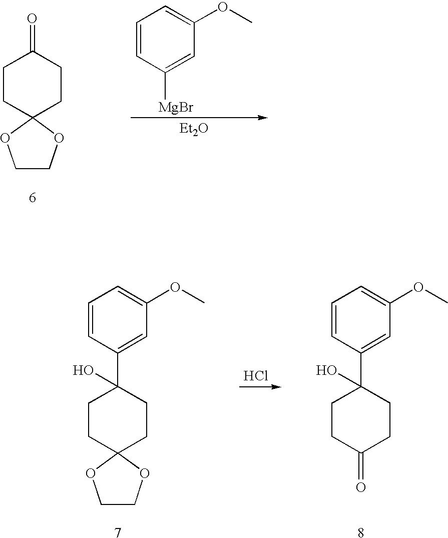

Magnesium chips (2.91 g, 0.12 mol) were covered with a layer of dry Et₂O (40 ml) and combined with approx. 1/3 of the m-bromoanisole to be used (22.44 g, 15.06 ml, 0.12 mol). Once the Grignard reaction had begun, the remaining m-bromoanisole dissolved in dry Et₂O (40 ml) was added dropwise such that the batch boiled gently. The mixture was then refluxed for a further 15 h. 1,4-Cyclohexanedione monoethylene ketal (15.62 g, 0.1 mol) dissolved in Et₂O (200 ml) was added dropwise to the solution, which had been cooled to 0° C., and stirred for 16 h. Working up was performed by pouring the reaction mixture into 2N HCl (100 ml) with ice cooling, separating the phases, extracting the aqueous phase with Et₂ (1×50 ml), washing the extract with water (3×50 ml) and drying it over sodium sulfate. Once the solvent had been removed by distillation, 4-hydroxy-4-(3′-methoxyphenyl)cyclohexan-1-one ethylene ketal (25.4 g) was obtained. The ketal was cleaved by dissolving the compound in THF (150 ml), adding 1N HCl (150 ml) with ice cooling and stirring the mixture for 16 h at room temperature. After addition of Et₂O (100ml), the phases were separated, the aqueous phase was extracted with Et₂O (1×50 ml), the organic phase washed with water (3×50 ml), dried over sodium sulfate and the solvent removed by distillation. The crude product was purified chromatographically (150 g silica gel, 3×1000 ml hexane/ethyl acetate 2:1). 13.89 g (63%) of the product could be obtained. The compound had a melting point of 105-108° C.

### Example 8

### Synthesis of 4-hydroxy-4-(3′-methoxyphenyl)cyclohexan-1-ol

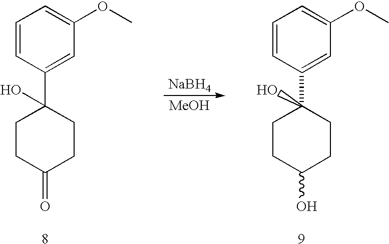

4-Hydroxy-4-(3′-methoxyphenyl)cyclohexan-1-one (3 g, 13.6 mmol) was dissolved in methanol (70 ml) and combined in portions with sodium borohydride (515 mg, 1.36 mmol). The reaction temperature should not exceed 30° C. during this operation. The mixture was stirred for 1 h at room temperature and then combined with water (20 ml). Methanol was removed by distillation, water (20 ml) was added, the mixture was extracted with dichloromethane (4×20 ml), dried and the solvent removed by distillation. 3.0 g (100%) of the product could be obtained. It remains to be clarified whether a mixture of diastereoisomers was formed and what configuration the diol formed has.

### Example 9

### Synthesis of 4-amino-1-(3′-methoxyphenyl)cyclohexan-1-ol

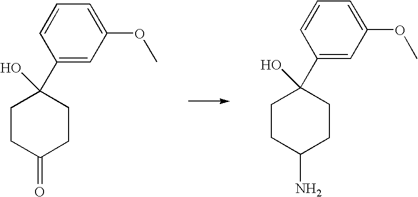

4-Hydroxy-4-(3′-methoxyphenyl)-cyclohexan-1-one (34 mmol, 7.5 g) was initially introduced under nitrogen in 225 ml THF and combined in an ice bath with ammonium trifluoroacetate (47 mmol, 6.23 g). After addition of 3 ml of glacial acetic acid, sodium triacetoxyborohydride (47 mmol, 10.05 g) was added in portions. Once addition was complete, the ice bath was removed and the reaction mixture stirred overnight at room temperature. After addition of 150 ml of 2N sodium hydroxide solution, the mixture was extracted three times with diethyl ether, washed with water and, after drying over sodium sulfate, evaporated. The crude product was purified on silica gel 60 with a mobile solvent mixture of methanol and 5% aqueous NH₄OH solution. After removal of the solvent, the product was obtained in the form of colourless crystals. 3.1 g (41% of theoretical) of product was obtained as a mixture of cis/trans isomers in a 3/1 ratio.

For the purposes of storage, the amine obtained may be precipitated as the hydrochloride.

### Example 10

### Synthesis of 4-amino-1-(3′-hydroxyphenyl)cyclohexan-1-ol

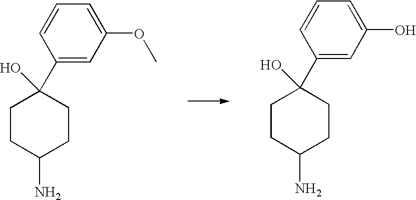

4-Amino-1-(3′-methoxyphenyl)-cyclohexan-1-ol (1.36 mmol, 300 mg) was dissolved in 6 ml of methanesulfonic acid, methionine (2 mmol, 303 mg) was added and the mixture stirred for 26 days at room temperature, wherein a clear solution was obtained. The mixture was combined with sodium carbonate with ice cooling, extracted with ethyl acetate and, after drying over sodium sulfate, evaporated. A colourless solid was obtained.

For the purposes of storage, the amine obtained may be precipitated as the hydrochloride.

### Pharmacological Investigations

Analgesic testing by writhing test in mice:

The in-depth investigation into analgesic efficacy was performed using phenylquinone-induced writhing in mice, as described above. The investigated compounds according to the invention exhibited an analgesic action. The results of selected writhing investigations are summarised in Table 1 below.

TABLE 1

| | % inhibition of writhing reactions |
|---|---|
| Example no. | 10 mg/kg i.v. |
| 1 | 72 |
| 2 | 55 |
| 3 | 90 |
| 4 | 84 |

1. Substituted 1H-quinoxalin-2-one compounds of the general formula I and the tautomers thereof, 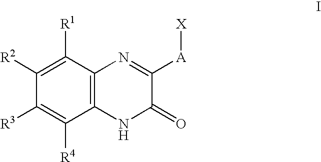
in which
R¹, R², R³ and R⁴, identical or different, denote a linear or branched, saturated or unsaturated aliphatic C₁₋₁₀ residue or a saturated or unsaturated cycloaliphatic C₃₋₇ residue, wherein each of the above-stated residues may optionally be joined together via an ether bridge, or hydrogen, a halogen or a hydroxy group,
A denotes a bridge with one of the following formulae: —(CH₂)ₙ₊₂—, —(CH₂)ₙ—CH═CH—, —(CH₂)ₙ—COO—, —(CH₂)ₙCONH—, —(CH₂)ₙ₊₁O(CH₂)ₚCO—, —(CH₂)ₙ₊₁, O—, —(CH₂)ₙ₊₁NR^{1′}—, —NH—(CH₂)ᵣ—, in which n denotes 0, 1, 2 or 3, p denotes 0 or 1 and r denotes 0, 1 or 2, R^{1′} has the meaning stated hereinafter and the bond to the residue X is always stated last and wherein bonding of the residues X¹⁷ and X¹⁸ is possible only via the three bridges stated first and bonding of the residue X⁷ via an amide bridge is excepted,
and X denotes one of the following residues of the general formulae X¹ to X¹⁶ and X¹⁸, in which the unoccupied bond line symbolises the bond to the bridge A and 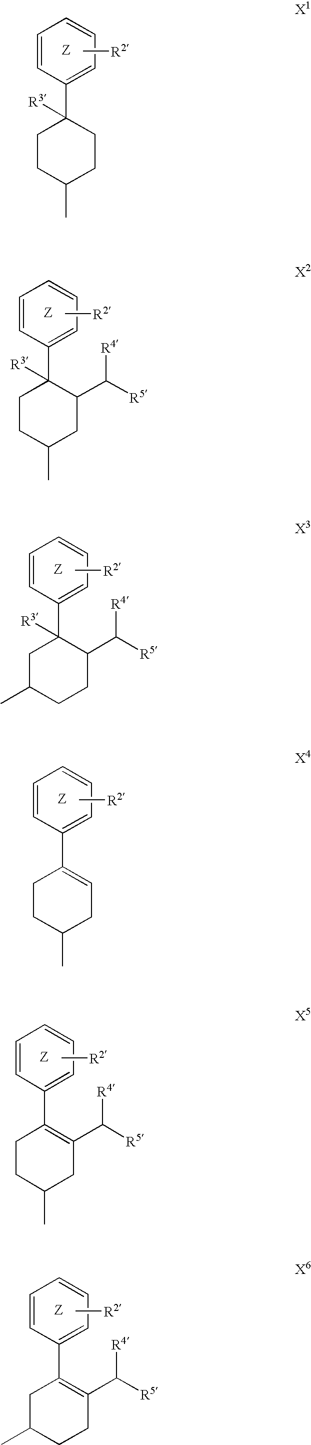 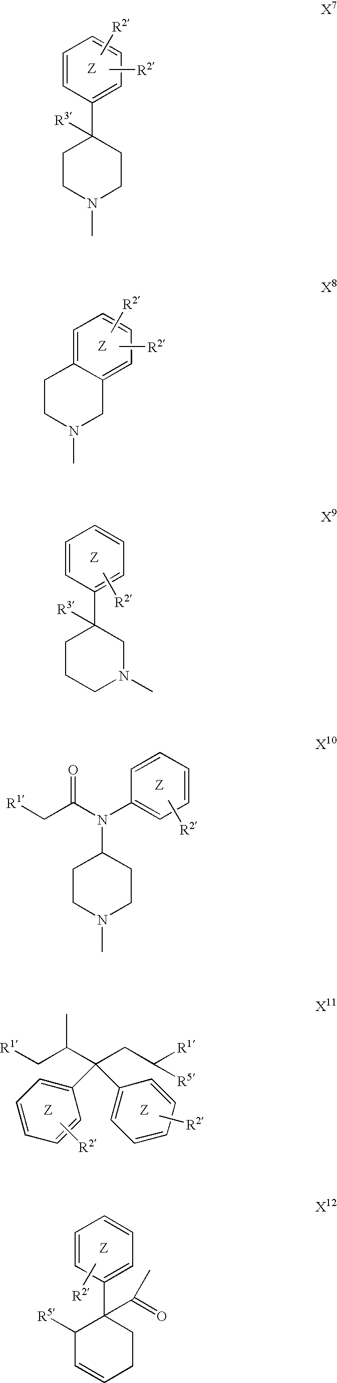 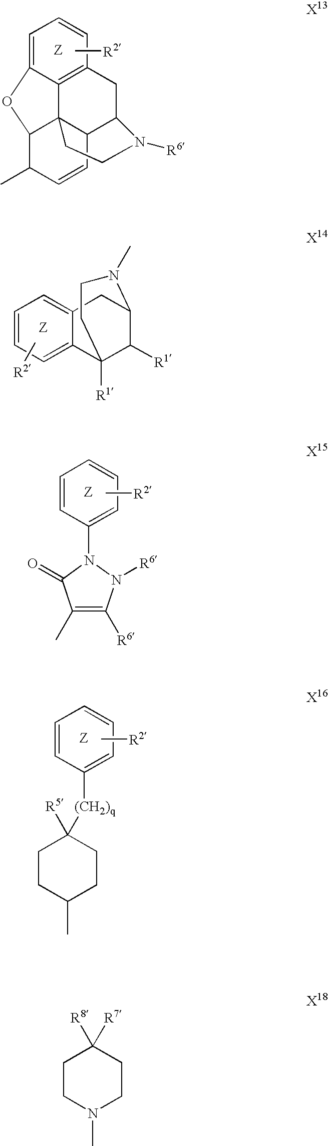
in which
R^{1′} denotes hydrogen, a linear or branched, saturated or unsaturated aliphatic C₁₋₁₀ residue, a saturated or unsaturated cycloaliphatic C₃₋₇ residue, an aryl or heteroaryl residue,
R^{2′} denotes a linear or branched, saturated or unsaturated aliphatic C₁₋₁₀ residue, a saturated or unsaturated cycloaliphatic C₃₋₇ residue or an aryl or heteroaryl residue, wherein all the above-stated residues may optionally be joined via an ether, thioether or SO₂ bridge, or hydrogen, a halogen, a hydroxy, thiol, cyano or nitro group or a group of the formula —CH₂F, —CHF₂, —CF₃ or —NR^{1′}₂, wherein the two residues R^{1′} are identical or different and have the above-stated meaning,
R^{3′} denotes a linear or branched, saturated or unsaturated aliphatic C₁₋₁₀ residue, a saturated or unsaturated cycloaliphatic C₃₋₇ residue, an aryl or heteroaryl residue, wherein all the above-stated residues may optionally be joined via an ether or an ester bridge, hydrogen, a halogen, a hydroxy group,
R^{4′} denotes hydrogen, an aryl or heteroaryl residue, wherein the aryl or heteroaryl residue may comprise at least one substituent R^{2′} with the above meaning, with the exception of hydrogen,
R^{5′} denotes a residue of the formula —NR^{6′}₂, wherein the two residues R^{6′} may be identical or different and have the meaning stated hereinafter or may form a 3-7-membered ring together with the nitrogen atom connecting them as a ring member, which ring may optionally contain at least one oxygen and/or at least one further nitrogen as a ring atom, wherein the nitrogen may comprise a substituent R^{10′} with the meaning stated hereinafter,
R^{6′} denotes a linear or branched, saturated or unsaturated aliphatic C₁₋₆ residue, a saturated or unsaturated cycloaliphatic C₃₋₇ residue, an aryl or heteroaryl residue,
R^{7′} denotes a cyano, amide or carboxylic acid residue,
R^{8′} denotes a residue of the formula —NR^{9′}₂, wherein the two residues R^{9′} may be identical or different and have the meaning stated hereinafter or may form a 3-7-membered ring together with the nitrogen atom connecting them as a ring member, which ring may optionally contain at least one oxygen and/or at least one further nitrogen as a ring atom,
R^{9′} denotes hydrogen, a linear or branched aliphatic C₁₋₁₀ residue,
R^{10′} denotes hydrogen, a linear or branched, saturated or unsaturated aliphatic C₁₋₁₀ residue, an aryl or heteroaryl residue and
Z denotes at least one optionally present oxygen, sulfur or nitrogen as a ring atom,
and q denotes 0, 1, 2 or 3,
optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

2. Substituted 1H-quinoxalin-2-one compounds and the tautomers thereof according to claim 1, characterised in that R² and R³, identical or different, denote a linear or branched, saturated or unsaturated aliphatic C₁₋₃ residue or a halogen and R¹ and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

3. Substituted 1H-quinoxalin-2-one compounds and the tautomers thereof according to claim 1, characterised in that R² and R³ in each case denote a methyl group or a chlorine and R¹ and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

4. Substituted 1H-quinoxalin-2-one compounds and the tautomers thereof according to claim 1, characterised in that R³ denotes a linear or branched, saturated or unsaturated aliphatic C₁₋₃ residue or a halogen and R¹, R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

5. Substituted 1H-quinoxalin-2-one compounds and the tautomers thereof according to claim 1, characterised in that R³ denotes a methyl group or a chlorine and R¹, R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

6. Substituted 1H-quinoxalin-2-one compounds and the tautomers thereof according to claim 1, characterised in that R¹ and R³, identical or different, denote a linear or branched, saturated or unsaturated aliphatic C₁₋₃ residue or a halogen and R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

7. Substituted 1H-quinoxalin-2-one compounds and the tautomers thereof according to claim 1, characterised in that R¹ and R³ in each case denote a methyl group or a chlorine and R² and R⁴ in each case denote hydrogen, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

8. Substituted 1H-quinoxalin-2-one compounds and the tautomers thereof according to claim 1, characterised in that A denotes a bridge of one of the following formulae: —CH₂—, —CH₂—CH₂—, —COO—, —(CH₂)ₙCONH—, wherein n denotes 0, 1 or 2, optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

9. Substituted 1H-quinoxalin-2-one compounds and the tautomers thereof according to claim 1, characterised in that X denotes a residue of the following formula: 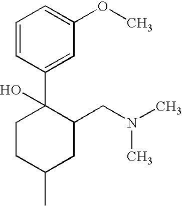
optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

10. Substituted 1H-quinoxalin-2-one compounds and the tautomers thereof according to claim 1:
6,7-Dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]amide,
6,7-Dichloro-3-oxo-3,4-dihydroquinoxaline 2-carboxylic acid [3′-(N,N-dimethylaminomethyl)-4′-hydroxy-4′-(m-methoxyphenyl)-cyclohexyl]amide,
6,7-Dimethyl-3-oxo-3,4-dihydroquinoxaline 2-carboxylic acid [3′-(N,N-dimethylaminomethyl)-4′-hydroxy-4′-(m-methoxyphenyl)-cyclohexyl] ester,
6,7-Dichloro-3-oxo-3,4-dihydroquinoxaline 2-carboxylic acid [3′-(N,N-dimethylaminomethyl)-4′-hydroxy-4′-(m-methoxyphenyl)-cyclohexyl] ester,
6,7-Dimethyl-3-oxo-3,4-dihydroquinoxaline 2-carboxylic acid [3′-(N,N-dimethylaminomethyl)-4′-hydroxy-4′-(m-methoxyphenyl)-cyclohexyl]propionamide,
6,7-Dichloro-3-oxo-3,4-dihydroquinoxaline 2-carboxylic acid [3′-(N,N-dimethylaminomethyl)-4′-hydroxy-4′-(m-methoxyphenyl)-cyclohexyl]propionamide,
optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

11. A process for the production of substituted 1H-quinoxalin-2-one compounds, the tautomers and corresponding stereoisomers thereof according to claim 1, characterised in that
A) an optionally substituted o-phenylenediamine of the general formula (1), in which R¹, R², R³ and R⁴ have the same meaning as in claim 1, is reacted 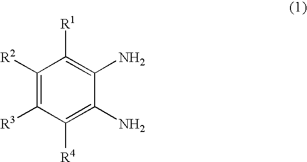
with a 2-ketodicarboxylic acid or a corresponding mono- or dialkyl ester of the general formula (2), in which R denotes a hydrogen or an alkyl group, preferably a methyl group or an ethyl group and n has the above-stated meaning, 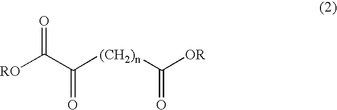
in the presence of an inorganic acid, preferably hydrochloric acid, in a suitable solvent at elevated temperature, preferably at 90-100° C., and is then worked up and the compound formed of the formula Y—COOR, in which R has the above-stated meaning and Y denotes a residue of the general formula Y, in which the unoccupied bond line symbolises the bond to the residue —COOR and 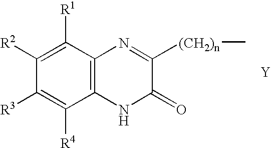
in which R¹, R², R³, R⁴ and n have the above-stated meaning, is optionally purified,
B) an optionally present ester of the formula Y—COOR, in which R denotes an alkyl group, preferably a methyl or ethyl group, is saponified in the presence of a base, preferably sodium or potassium hydroxide, in a suitable solvent, preferably an alcohol/water mixture, particularly preferably in a methanol or ethanol/water mixture, and is then worked up and the carboxylic acid formed of the formula Y—COOH is optionally purified,
C) a carboxylic acid or a carboxylic acid ester of the formula Y—COOR, in which Y has the above-stated meaning and R denotes hydrogen or an alkyl group, preferably a methyl or ethyl group, is optionally derivatised in that
a) a carboxylic acid or a carboxylic acid ester of the formula Y—COOR is reduced with the assistance of reducing agents, preferably lithium aluminium hydride, in a suitable solvent, preferably tetrahydrofuran, to yield the corresponding alcohol of the formula Y—CH₂—OH,
b) a carboxylic acid or carboxylic acid ester of the formula Y—COOR is reduced with the assistance of reducing agents, preferably diisobutylaluminium hydride, in a suitable solvent, preferably hexane, to yield the corresponding aldehyde of the formula Y—CHO,
c) an alcohol of the formula Y—CH₂—OH according to a) is reacted with a brominating agent, preferably PBr₃ or Ph₃PBr₂ to yield the corresponding bromide of the formula Y—CH₂-Br or
d) a carboxylic acid of the formula Y—COOH, wherein in the above-stated formula Y n denotes 0 is reacted firstly with (PhO)₂—P(O)—N₃ in a suitable solvent at elevated temperature and then with water to yield the corresponding amine of the formula Y—NH₂
and is then worked up and the product is optionally purified,
D) a compound of the formula X—R′, in which X has the above-stated meaning and R′ denotes a functional group, is optionally derivatised in that
a) a ketone of the formula X═O is reacted 1) with methoxymethyl triphenylphosphinium chloride under protective gas in a suitable solvent, preferably in dimethylformamide, in the presence of sodium hydride and then with hydrochloric acid or 2) with Me₃S⁺BF₄⁻ to yield the corresponding aldehyde X—CHO extended by one carbon atom,
b) an aldehyde of the formula X—CHO according to a) is reacted with a reducing agent, preferably sodium borohydride, in a suitable solvent, preferably an ethanol/water mixture, to yield the corresponding alcohol X—CH₂—OH,
c) an alcohol X—CH₂—OH according to b) or of the formula X—OH is reacted with a brominating agent, preferably triphenylphosphine dibromide, in a suitable solvent, preferably acetonitrile, to yield the corresponding bromide of the formula X—CH₂—Br or X—Br,
d) a bromide of the formula X—CH₂—Br according to c) is reacted with a phosphine of the formula PR″₃, in which R″ denotes an organic residue, preferably a phenyl residue, in a suitable solvent, preferably toluene, ether, tetrahydrofuran or acetone, with cooling and under protective gas to yield the corresponding phosphonium salt R″₃P⁺—CHX⁻,
e) a bromide of the formula X—CH₂—Br according to c) is reacted with a phosphite of the formula HP(O) (OR″′)₂, in which R″′ denotes an organic residue, at elevated temperature, preferably 200° C., to yield the corresponding phosphonate (R″′O)2P(O)—CH₂—X
and is then worked up and the product is optionally purified,
E) a compound from step A), B) or C), in which Y has the above-stated meaning, is reacted with a compound from step D) or a compound of the formula X—R′, in which X and R′ have the above-stated meaning, in that
a) a carboxylic acid of the formula Y—COOH is reacted with an amine of the formula X—NH₂ in the presence of a suitable condensing agent, preferably dicyclohexyl carbodiimide, 1-hydroxybenzotriazole and N-methylmorphine, in a suitable solvent, preferably dimethylformamide, with formation of an amide bridge,
b) a carboxylic acid of the formula Y—COOH is reacted with an alcohol of the formula X—OH in the presence of a suitable condensing agent in a suitable solvent with formation of an ester bridge, the reaction preferably taking place in the presence of methylimidazole and 1-(mesitylene-2′-sulfonyl)-3-nitro-1,2,4-triazole in tetrahydrofuran or in the presence of dicyclohexylcarbodiimide, 1-hydroxybenzotriazole and N-methylmorphine in dimethylformamide,
c) a bromide of the formula Y—CH₂—Br is reacted with a compound of the formula X—CO(CH₂)ₚ—OH, in which p has the above-stated meaning, under protective gas in the presence of a suitable catalyst, preferably sodium hydride or potassium tert-butylate, in a suitable solvent, preferably dimethylformamide, with formation of a bridge of the formula —CO(CH₂)ₚ—O—CH₂,
d) an alcohol of the formula Y—CH₂—OH is reacted with a bromide of the formula X—Br under protective gas in the presence of a suitable condensing agent, preferably sodium hydride or potassium tert-butylate, in a suitable solvent, preferably dimethylformamide, with formation of an ether bridge,
e) a bromide of the formula Y—CH₂—Br is reacted with an alcohol of the formula X—OH under protective gas in the presence of a suitable condensing agent, preferably sodium hydride or potassium tert-butylate, in a suitable solvent, preferably dimethylformamide, with formation of an ether bridge,
f) an aldehyde of the formula Y—CHO is reacted with an amine of the formula X—NHR^{1′} in the presence of a suitable reducing agent, preferably sodium cyanoborohydride and sodium triacetoxyborohydride, in a suitable solvent, preferably a mixture of tetrahydrofuran and 1,2-dichloroethane, with formation of an amino bridge,
g) an amine of the formula Y—NH₂, wherein in the above-stated formula Y n denotes 0, is reacted with a bromide of the formula X—(CH₂)ᵣBr in the presence of a suitable catalyst, preferably caesium carbonate, in a suitable solvent, preferably dimethylformamide, with formation of an —NH—(CH₂)ᵣ— bridge,
h) an aldehyde of the formula Y—CHO is reacted with a phosphonium salt R″₃P⁺—CHX⁻, in which R″ has the above-stated meaning, under protective gas in the presence of suitable catalysts in a suitable solvent, preferably in the presence of sodium methanolate in a mixture of hexane, diethyl ether and/or diisopropyl ether or in the presence of sodium hydride, potassium tert-butylate or a lithium amide in dimethylformamide or dimethyl sulfoxide, with formation of a —CH═CH— bridge or
i) an aldehyde of the formula Y—CHO is reacted with a phosphonate of the formula (R″′O)₂P(O)—CH₂—X, in which R″′ has the above-stated meaning, under protective gas in the presence of suitable catalysts, preferably sodium methanolate, sodium hydroxide, potassium hydroxide, sodium hydride, potassium tert-butylate or a lithium amide, in a suitable solvent, preferably dimethylformamide, dimethyl sulfoxide, diethyl ether, tetrahydrofuran, with formation of a —CH═CH— bridge and
j) optionally the —CH═CH— bridge from step h) or i) is hydrogenated by hydrogen, preferably at standard pressure or elevated pressure of up to 100 bar, in the presence of suitable catalysts, preferably transition metals or transition metal compounds, preferably palladium or the salts thereof, rhodium or the complexes thereof, in a suitable solvent, preferably dimethylformamide, methanol or ethanol, at a temperature of between 20 and 100° C. with formation of a —CH₂—CH₂— bridge
and is then worked up and the product is optionally purified.

12. A pharmaceutical preparation containing at least one substituted 1H-quinoxalin-2-one compound or the tautomer thereof optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, according to claim 1 and optionally physiologically acceptable auxiliary substances.

13. A pharmaceutical preparation according to claim 12 for combatting pain.

14. A pharmaceutical preparation according to claim 13 for combatting chronic pain.

15. A pharmaceutical preparation according to claim 13 for combatting neuropathic pain.

16. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of neurodegenerative diseases, preferably Alzheimer's disease, Parkinson's disease or Huntington's chorea.

17. A pharmaceutical preparation according to claim 12 for the treatment or prevention of stroke.

18. A pharmaceutical preparation according to claim 12 for the treatment or prevention of cerebral ischaemia.

19. A pharmaceutical preparation according to claim 12 for the treatment or prevention of cerebral infarct.

20. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of cerebral oedema.

21. A pharmaceutical preparation according to claim 12 for the treatment or prevention of insufficiency states of the central nervous system, preferably hypoxia or anoxia.

22. A pharmaceutical preparation according to claim 12 for the treatment or prevention of epilepsy.

23. A pharmaceutical preparation according to claim 12 for the treatment or prevention of schizophrenia.

24. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of psychoses brought about by elevated amino acid levels.

25. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of AIDS dementia.

26. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of Tourette's syndrome.

27. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of encephalomyelitis.

28. A pharmaceutical preparation according to claim 12 for the treatment or prevention of perinatal asphyxia.

29. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of tinnitus.

30. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of migraine.

31. A pharmaceutical preparation containing at least one substituted 1H-quinoxalin-2-one compound or the tautomer thereof according to claim 1 and/or at least one substituted 1H-quinoxalin-2-one compound or the tautomer thereof according to claims 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of inflammatory and/or allergic reactions.

32. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of depression.

33. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of mental health conditions.

34. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of urinary incontinence.

35. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of pruritus.

36. A pharmaceutical preparation containing at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for the treatment or prevention of diarrhoea.

37. A pharmaceutical preparation containing at least one substituted 1H-quinoxalin-2-one compound or the tautomer thereof according to claim 1 and/or at least one substituted 1H-quinoxalin-2-one compound or the tautomer thereof according to claims 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, and optionally physiologically acceptable auxiliary substances for anxiolysis.

38. A pharmaceutical preparation according to claim 12 for anaesthesia.

39. Use of at least one substituted 1H-quinoxalin-2-one compound or the tautomer thereof optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, according to claim 1 for the production of a pharmaceutical preparation for combatting pain, preferably chronic or neuropathic pain.

40. Use of at least one substituted 1H-quinoxalin-2-one compound or the tautomer thereof, optionally in the form of the racemate thereof, the pure stereoisomer thereof, in particular enantiomer or diastereomer, or in the form of mixtures of the stereoisomers, in particular the enantiomers or diastereomers, in any desired mixing ratio, or in each case in the form of the acid or base thereof or in the form of the salt thereof, in particular of a physiologically acceptable salt, or in the form of the solvate thereof, in particular the hydrate, according to claim 1 for the production of a pharmaceutical preparation for the treatment or prevention of stroke, neurodegenerative diseases, preferably Alzheimer's disease, Parkinson's disease or Huntington's chorea, for the treatment or prevention of cerebral ischaemia, cerebral infarct, insufficiency states of the central nervous system, preferably hypoxia or anoxia, epilepsy, schizophrenia, perinatal asphyxia or for anaesthesia.

41. Use of at least one substituted 1H-quinoxalin-2-one compound or the tautomer thereof according to claim 1 and/or at least one substituted 1H-quinoxalin-2-one compound or the tautomer thereof according to claim 1, in which the residue X⁷ is attached via an amide bridge, in each case optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates, for the production of a pharmaceutical preparation for the treatment or prevention of cerebral oedema, psychoses brought about by elevated amino acid levels, AIDS dementia, encephalomyelitis, Tourette's syndrome, tinnitus, migraine, inflammatory and/or allergic reactions, depression, mental health conditions, urinary incontinence, pruritus or diarrhoea or for anxiolysis.

42. Substituted 4-aryl- and 4-heteroarylcyclohexane compounds of the general formula II, 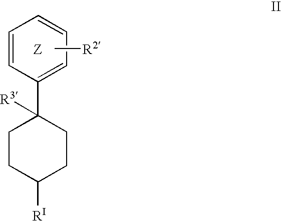
in which
R^{I} denotes a keto or aldehyde group or a group of the formula —NHR^{1′}, —CO—(CH₂)ₚ—OH, —(CH₂)ᵣOH or —(CH₂)ᵣBr, wherein R^{1′} has the meaning stated hereinafter and p denotes 0 or 1 and r denotes 0, 1 or 2,
R^{1′} denotes hydrogen, a linear or branched, saturated or unsaturated aliphatic C₁₋₁₀ residue, a saturated or unsaturated cycloaliphatic C₃₋₇ residue, an aryl or heteroaryl residue,
R^{2′} denotes a linear or branched, saturated or unsaturated aliphatic C₁₋₁₀ residue, a saturated or unsaturated cycloaliphatic C₃₋₇ residue or an aryl- or heteroaryl residue, wherein all the above-stated residues may optionally be joined via an ether, thioether or SO₂ bridge, or hydrogen, a halogen, a hydroxy, thiol, cyano or nitro group or a group of the formula —CH₂F, —CHF₂, —CF₃ or —NR^{1′}₂, wherein the two residues R^{1′} are identical or different and have the above-stated meaning,
R^{3′} denotes a linear or branched, saturated or unsaturated aliphatic C₁₋₁₀ residue, a saturated or unsaturated cycloaliphatic C₃₋₇ residue, an aryl or heteroaryl residue, wherein all the above-stated residues may optionally be joined via an ether or an ester bridge, a halogen and
Z denotes at least one optionally present oxygen, sulfur or nitrogen as a ring atom,
optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates,
with the exception of cis-4-amino-1-phenylcyclohexanol, trans-4-ethanoyl-1-phenylcyclohexane and trans-4-butanoyl-1-phenylcyclohexane.

43. A substituted 4-aryl- and 4-heteroarylcyclohexane compound according to claim 42:
4-Amino-4-(3′-methoxyphenyl)cyclohexan-1-ol,
4-Amino-4-(3′-hydroxyphenyl)cyclohexan-1-ol,
optionally in the form of the racemates thereof, the pure stereoisomers thereof, in particular enantiomers or diastereomers, or in the form of mixtures of the stereoisomers, in particular the enantiomers or diastereomers, in any desired mixing ratio or in each case in the form of the acids or bases thereof or in the form of the salts thereof, in particular physiologically acceptable salts, or in the form of the solvates thereof, in particular the hydrates.

44. A process for the production of substituted 4-aryl- and 4-heteroarylcyclohexane compounds according to claim 41, in which
a) 1,4-cyclohexanedione monoethylene ketal, 4-aminocyclohexan-1-one ethylene ketal or 4-oxocyclohexanecarboxylic acid is reacted with magnesium and a brominated or chlorinated, optionally substituted aromatic or heteroaromatic compound in a suitable solvent, preferably dry diethyl ether, at elevated temperature to yield the corresponding coupling product and then the ketal is optionally cleaved by reaction with hydrochloric acid in a suitable solvent, preferably tetrahydrofuran, and worked up, optionally followed by purification of the product of the formula X^{1a}═O, X^{1a}—NHR^{1′} or X^{1a}—CO₂H, in which X^{1a} denotes a residue of the formula X^{1a} and R^{1′}, R^{2′} and Z have the above-stated meaning and the unoccupied bond line symbolises the bond to the respective residue ═O, —NHR^{1′} or —CO₂H, 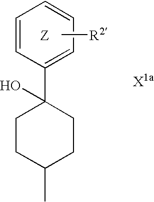
b) a ketone of the formula X^{1a}═O is optionally reacted in the presence of a suitable reducing agent, preferably sodium borohydride, in a suitable solvent, preferably methanol, to yield the corresponding alcohol of the formula X^{1a}—OH, is worked up and the product is optionally purified,
c) a ketone of the formula X^{1a}═O is optionally reacted under nitrogen in a suitable solvent, preferably tetrahydrofuran, firstly with ammonium trifluoroacetate and then with glacial acetic acid and sodium triacetoxyborohydride, to yield the corresponding amine of the formula X^{1a}—NH₂, is worked up and the product is optionally purified,
d) a carboxylic acid of the formula X^{1a}—CO₂H is optionally activated by reaction with dicyclohexylcarbodiimide or by conversion into the carboxylic acid chloride or a mixed anhydride, is reacted with diazomethane in a suitable solvent, preferably ether, and is then treated with water, worked up and the product of the formula X^{1a}—CO—CH₂—OH is optionally purified,
e) a compound from step d) is optionally reacted firstly in the presence of a suitable reducing agent in a suitable solvent to yield a compound of the formula X^{1a}—(CH₂)₂—OH and then this compound is reacted with a brominating agent, preferably PPh₃/Br₂, in a suitable solvent to yield a compound of the formula X^{1a}—(CH₂)₂—Br, is worked up and the product is optionally purified,
f) a ketone of the formula X^{1a}═O according to a) is reacted 1) with methoxymethyl triphenylphosphinium chloride under protective gas in a suitable solvent, preferably in dimethylformamide, in the presence of sodium hydride and then with hydrochloric acid or 2) with Me₃S⁺BF₄⁻ to yield the corresponding aldehyde X^{1a}—CHO extended by one carbon atom, is then worked up and the product is optionally purified,
g) an aldehyde of the formula X^{1a}—CHO according to f) is reacted with a reducing agent, preferably sodium borohydride, in a suitable solvent, preferably an ethanol/water mixture to yield the corresponding alcohol X^{1a}—CH₂—OH, is then worked up and the product is optionally purified,
h) an alcohol of the formula X^{1a}—CH₂—OH according to g) or of the formula X^{1a}—OH according to b) is reacted with a brominating agent, preferably triphenylphosphine dibromide, in a suitable solvent, preferably acetonitrile, to yield the corresponding bromide of the formula X^{1a}—CH₂—Br or X^{1a}—Br respectively, is then worked up and the product is optionally purified,
i) the hydroxy group in position 4 of the cyclohexane ring in the residue X^{1a} is optionally converted into hydrogen, a halogen, an ether, ester, aryl or heteroaryl group or into an aliphatic or cycloaliphatic residue, in that
α) in order to introduce an ether group, a compound from one of steps a)-h) is reacted with an aliphatic or cycloaliphatic compound in the presence of a suitable catalyst in a suitable solvent, preferably in the presence of sodium hydride in dimethylformamide or in the presence of potassium hydroxide in dimethyl sulfoxide, or with an alkylating agent in a suitable solvent, preferably with a diazo compound in diethyl ether, or with an aryl or heteroaryl compound in the presence of diethylazo dicarboxylate and triphenylphosphine,
β) in order to introduce a halogen, a compound from one of steps a)-h) is reacted with a halogenating agent in a suitable solvent, preferably with POCl₃ in dimethylformamide, with PPh₃/Cl₂, with PPh₃/Br₂, with triphenylphosphine/n-chlorosuccinimide or with HCl/ZnCl₂,
γ) in order to introduce hydrogen, a compound from step β) is reacted with hydrogen in the presence of a suitable catalyst, preferably palladium/carbon, in a suitable solvent,
δ) in order to introduce an aliphatic or cycloaliphatic residue, or aryl or heteroaryl group, a compound from step β) is reacted with an aliphatic or cycloaliphatic boronic acid or a boronic acid ester or an aryl or heteroaryl borodihydroxide compound in the presence of palladium(II) acetate and potassium carbonate in a suitable solvent, preferably a dimethylformamide/water mixture, or
ε) in order to introduce an ester group, a compound from one of steps a)-h) is reacted with a carboxylic acid chloride in the presence of a suitable catalyst in a suitable solvent
and is then worked up, optionally followed by purification of the compound formed of the formula X¹-R′, in which X¹ denotes the formula X¹ 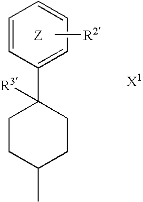
and R^{I}, R^{2′} and R^{3′} have the above-stated meaning.

45. A process for the production of substituted 4-aryl- and 4-heteroarylcyclohexane compounds according claim 41, in which
a) 1,4-cyplohexanedione monoethylene ketal, 4-aminocyclohexan-1-one ethylene ketal or 4-oxocyclohexanecarboxylic acid is reacted with magnesium and a brominated or chlorinated, optionally substituted aromatic or heteroaromatic compound in a suitable solvent, preferably dry diethyl ether, at elevated temperature to yield the corresponding coupling product and then the ketal is optionally cleaved by reaction with hydrochloric acid in a suitable solvent, preferably tetrahydrofuran and worked up, optionally followed by purification of the product of the formula X^{1a}═O, X^{1a}—NHR^{1′} or X^{1a}—CO₂H, in which X^{1a} denotes a residue of the formula X^{1a} and R^{1′}, R^{2′} and Z have the above-stated meaning and the unoccupied bond line symbolises the bond to the respective residue ═O, —NHR^{1′} or —CO₂H, 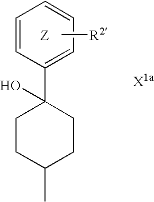
b) a ketone of the formula X^{1a}═O is optionally reacted in the presence of a suitable reducing agent, preferably sodium borohydride, in a suitable solvent, preferably methanol, to yield the corresponding alcohol of the formula X^{1a}—OH, is worked up and the product is optionally purified,
c) a ketone of the formula X^{1a}═O is optionally reacted under nitrogen in a suitable solvent, preferably tetrahydrofuran, firstly with ammonium trifluoroacetate and then with glacial acetic acid and sodium triacetoxyborohydride, to yield the corresponding amine of the formula X^{1a}—NH₂, is worked up and the product is optionally purified,
d) a carboxylic acid of the formula X^{1a}—CO₂H is optionally activated by reaction with dicyclohexylcarbodiimide or by conversion into the carboxylic acid chloride or a mixed anhydride, is reacted with diazomethane in a suitable solvent, preferably ether, and is then treated with water, worked up and the product of the formula X^{1a}—CO—CH₂—OH is optionally purified,
e) a compound from step d) is optionally reacted firstly in the presence of a suitable reducing agent in a suitable solvent to yield a compound of the formula X^{1a}—(CH₂)₂—OH and then this compound is reacted with a brominating agent, preferably PPh₃/Br₂, in a suitable solvent to yield a compound of the formula X^{1a}—(CH₂)₂—Br, is worked up and the product is optionally purified,
f) a ketone of the formula X^{1a}═O according to a) is reacted 1) with methoxymethyl triphenylphosphinium chloride under protective gas in a suitable solvent, preferably in dimethylformamide, in the presence of sodium hydride and then with hydrochloric acid or 2) with Me₃S⁺BF₄⁻ to yield the corresponding aldehyde X^{1a}—CHO extended by one carbon atom, is then worked up and the product is optionally purified,
g) an aldehyde of the formula X^{1a}—CHO according to f) is reacted with a reducing agent, preferably sodium borohydride, in a suitable solvent, preferably an ethanol/water mixture to yield the corresponding alcohol X^{1a}—CH₂—OH, is then worked up and the product is optionally purified,
h) an alcohol of the formula X^{1a}—CH₂—OH according to g) or of the formula X^{1a}—OH according to b) is reacted with a brominating agent, preferably triphenylphosphine dibromide, in a suitable solvent, preferably acetonitrile, to yield the corresponding bromide of the formula X^{1a}—CH₂—Br or X^{1a}—Br respectively, is then worked up and the product is optionally purified,
i) the hydroxy group in position 4 of the cyclohexane ring in the residue X^{1a} is optionally converted into hydrogen, a halogen, an ether, ester, aryl or heteroaryl group or into an aliphatic or cycloaliphatic residue, in that
α) in order to introduce an ether group, a compound from one of steps a)-h) is reacted with an aliphatic or cycloaliphatic compound in the presence of a suitable catalyst in a suitable solvent, preferably in the presence of sodium hydride in dimethylformamide or in the presence of potassium hydroxide in dimethyl sulfoxide, or with an alkylating agent in a suitable solvent, preferably with a diazo compound in diethyl ether, or with an aryl or heteroaryl compound in the presence of diethylazo dicarboxylate and triphenylphosphine,
β) in order to introduce a halogen, a compound from one of steps a)-h) is reacted with a halogenating agent in a suitable solvent, preferably with POCl₃ in dimethylformamide, with PPh₃/Cl₂, with PPh₃/Br₂, with triphenylphosphine/n-chlorosuccinimide or with HCl/ZnCl₂,
γ) in order to introduce hydrogen, a compound from step β) is reacted with hydrogen in the presence of a suitable catalyst, preferably palladium/carbon, in a suitable solvent,
δ) in order to introduce an aliphatic or cycloaliphatic residue, or aryl or heteroaryl group, a compound from step β) is reacted with an aliphatic or cycloaliphatic boronic acid or a boronic acid ester or an aryl or heteroaryl borodihydroxide compound in the presence of palladium(II) acetate and potassium carbonate in a suitable solvent, preferably a dimethylformamide/water mixture, or
ε) in order to introduce an ester group, a compound from one of steps a)-h) is reacted with a carboxylic acid chloride in the presence of a suitable catalyst in a suitable solvent
and is then worked up, optionally followed by purification of the compound formed of the formula X¹-R′, in which X¹ denotes the formula X¹ 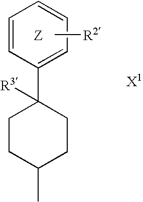
and R^{I}, R^{2′} and R^{3′} have the above-stated meaning.
